SEQUENCE LISTING (52 sequences)

SEQ ID NO: 1 - DNA, 369 bp, Synthetic construct
  source 1..369: mol_type = other DNA
caggttcagctgcagcagtctggggctgagctggtgaagcctggggcctcagtgaagatttcctgcaaagcttctggctacgcattcagtgactactggatgaactgggtgaagcagaggcctggaaagggtcttgagtggattggacagatttatcctggagatgatgatactaactacaacggaaagttcaagggcaaggccacactgactgcagacaaatcctccagcacagcctacatgcagctcagcagcctgacctctgaggactctgcggtctatttctgtgcaagagagggttactacggttatacctacgtggagtacttcgatgtctggggcacagggaccacggtcaccgtctcctca

SEQ ID NO: 2 - protein, 123 aa, Synthetic construct
QVQLQQSGAELVKPGASVKISCKASGYAFSDYWMNWVKQRPGKGLEWIGQIYPGDDDTNYNGKFKGKATLTADKSSSTAYMQLSSLTSEDSAVYFCAREGYYGYTYVEYFDVWGTGTTVTVSS

SEQ ID NO: 3 - DNA, 318 bp, Synthetic construct
  source 1..318: mol_type = other DNA
caaattgttctcacccagtctccagcaatcatgtctgtatctccaggggagaaggtcaccttgacctgcagcgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggactagtaacccactcacgttcggtgctgggaccaagctggagctgaga

SEQ ID NO: 4 - protein, 106 aa, Synthetic construct
QIVLTQSPAIMSVSPGEKVTLTCSASSSVSYMHWYQQKSGTSPKRWIYDTSKLASGVPARFSGSGSGTSYSLTISSMEAEDAATYYCQQWTSNPLTFGAGTKLELR

SEQ ID NO: 5 - DNA, 369 bp, Synthetic construct
  source 1..369: mol_type = other DNA
caagtgcagctgcagcagagcggcgccgagctggtgaagcctggcgctagcgtgaagatcagctgcaaggctagcggctacgccttcagcgactactggatgaactgggtgaagcagagacctggcaagggcctggagtggatcggacagatctaccctggcgacgacgacaccaactacaacggcaagttcaagggcaaggccaccctgaccgccgacaagagcagcagcaccgcctacatgcagctgagcagcctgacaagcgaggacagcgccgtgtacttctgcgctagagagggctactacggctacacctacgtggagtacttcgacgtgtggggcaccggcaccaccgtgaccgtgagcagc

SEQ ID NO: 6 - protein, 123 aa, Synthetic construct; identical to SEQ ID NO: 2

SEQ ID NO: 7 - DNA, 318 bp, Synthetic construct
  source 1..318: mol_type = other DNA
cagatcgtgctgacacagagccctgccatcatgagcgtgagccctggcgagaaggtgaccctgacctgcagcgctagcagcagcgtgagctacatgcactggtatcagcagaagtccggcacaagccctaagagatggatctacgacacaagcaagctggctagcggcgtgcctgctagattcagcggcagcggcagcggcacaagctacagcctgaccatcagcagcatggaggccgaggacgccgccacctactactgtcagcagtggacaagcaaccctctgaccttcggcgccggcaccaagctggagctgaga

SEQ ID NO: 8 - protein, 106 aa, Synthetic construct; identical to SEQ ID NO: 4

SEQ ID NO: 9 - protein, 20 aa, Synthetic construct
MEAPAQLLFLLLLWLPDTTG

SEQ ID NO: 10 - protein, 327 aa, Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 107 aa, Synthetic construct [sequence omitted]

SEQ ID NO: 12 - protein, 103 aa, Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 13 - protein, 123 aa, Synthetic construct
QVQLVQSGAEVKKPGESLKISCKASGYAFSDYWINWVRQMPGKGLEWMGQIYPGDDDTNYNPSFQGQVTISADKSISTAYLQWSSLKASDTAMYYCAREGYYGYTYVEYFDVWGTGTTVTVSS

SEQ ID NO: 14 - protein, 8 aa, Synthetic construct
GYAFSDYW

SEQ ID NO: 15 - protein, 8 aa, Synthetic construct
IYPGDDDT

SEQ ID NO: 16 - protein, 16 aa, Synthetic construct
AREGYYGYTYVEYFDV

SEQ ID NO: 17 - protein, 106 aa, Synthetic construct
QIVLTQSPATLSLSPGERATLSCRASSSVSYLHWYQQKPGQSPRLWIYDTSKRATGIPARFSGSGSGTDYTLTISSLEPEDFAVYYCQQWTSNPLTFGQGTKLEIK

SEQ ID NO: 18 - protein, 5 aa, Synthetic construct
SSVSY

SEQ ID NO: 19 - skipped

SEQ ID NO: 20 - protein, 9 aa, Synthetic construct
QQWTSNPLT

SEQ ID NO: 21 - protein, 123 aa, Synthetic construct; identical to SEQ ID NO: 13

SEQ ID NO: 22 - protein, 8 aa, Synthetic construct
GYAFSDYW

SEQ ID NO: 23 - protein, 8 aa, Synthetic construct
IYPGDDDT

SEQ ID NO: 24 - protein, 16 aa, Synthetic construct
AREGYYGYTYVEYFDV

SEQ ID NO: 25 - protein, 106 aa, Synthetic construct
EIVLTQSPATLSLSPGERATLSCRASSSVSYLHWYQQKPGQSPRLWIYDTSKRATGIPARFSGSGSGTDYTLTISSLEPEDFAVYYCQQWTSNPLTFGQGTKLEIK

SEQ ID NO: 26 - protein, 5 aa, Synthetic construct
SSVSY

SEQ ID NO: 27 - skipped

SEQ ID NO: 28 - protein, 9 aa, Synthetic construct
QQWTSNPLT

SEQ ID NO: 29 - protein, 123 aa, Synthetic construct
QVQLVQSGAEVKKPGESLKISCKGSGYAFSDYWINWVRQMPGKGLEWMGQIYPGDDDTNYNPSFQGQATLSADKSISTAYLQWSSLKASDTAMYYCAREGYYGYTYVEYFDVWGTGTTVTVSS

SEQ ID NO: 30 - protein, 8 aa, Synthetic construct
GYAFSDYW

SEQ ID NO: 31 - protein, 8 aa, Synthetic construct
IYPGDDDT

SEQ ID NO: 32 - protein, 16 aa, Synthetic construct
AREGYYGYTYVEYFDV

SEQ ID NO: 33 - protein, 106 aa, Synthetic construct; identical to SEQ ID NO: 25

SEQ ID NO: 34 - protein, 5 aa, Synthetic construct
SSVSY

SEQ ID NO: 35 - skipped

SEQ ID NO: 36 - protein, 9 aa, Synthetic construct
QQWTSNPLT

SEQ ID NO: 37 - protein, 123 aa, Synthetic construct
QVQLVQSGAEVKKPGESLKISCKASGYAFSDYWINWVRQMPGKGLEWMGQIYPGDDDTNYNPSFQGQATLSADKSISTAYLQWSSLKASDTAMYYCAREGYYGYTYVEYFDVWGTGTTVTVSS

SEQ ID NO: 38 - protein, 8 aa, Synthetic construct
GYAFSDYW

SEQ ID NO: 39 - protein, 7 aa, Synthetic construct
YPGDDDT

SEQ ID NO: 40 - protein, 16 aa, Synthetic construct
AREGYYGYTYVEYFDV

SEQ ID NO: 41 - protein, 106 aa, Synthetic construct; identical to SEQ ID NO: 17

SEQ ID NO: 42 - protein, 5 aa, Synthetic construct
SSVSY

SEQ ID NO: 43 - skipped

SEQ ID NO: 44 - protein, 9 aa, Synthetic construct
QQWTSNPLT

SEQ ID NO: 45 - protein, 123 aa, Synthetic construct; identical to SEQ ID NO: 29

SEQ ID NO: 46 - protein, 8 aa, Synthetic construct
GYAFSDYW

SEQ ID NO: 47 - protein, 8 aa, Synthetic construct
IYPGDDDT

SEQ ID NO: 48 - protein, 16 aa, Synthetic construct
AREGYYGYTYVEYFDV

SEQ ID NO: 49 - protein, 106 aa, Synthetic construct; identical to SEQ ID NO: 17

SEQ ID NO: 50 - protein, 5 aa, Synthetic construct
SSVSY

SEQ ID NO: 51 - skipped

SEQ ID NO: 52 - protein, 9 aa, Synthetic construct
QQWTSNPLT

(19) United States
(12) Patent Application Publication
(10) Pub. No.: US 2026/0125455 A1
(43) Pub. Date: May 7, 2026

(54) HUMAN C6 BINDING MOLECULES FOR TREATING DISEASES

(71) Applicant: The Cleveland Clinic Foundation, Cleveland, OH (US)

(72) Inventor: Feng LIN, Cleveland, OH (US)

(21) Appl. No.: 19/122,997

(22) PCT Filed: Oct. 20, 2023
(86) PCT No.: PCT/US2023/077377

§ 371 Date: Apr. 21, 2025

Related U.S. Application Data
(60) Provisional application No. 63/380,391, filed on Oct. 21, 2022.

(51) Int. Cl. C07K 16/18; A61K 39/00; A61P 7/04

(52) U.S. Cl. CPC C07K 16/18; A61P 7/04; A61K 2039/505; C07K 2317/24; C07K 2317/33; C07K 2317/92

(57) ABSTRACT

Provided herein are human C6 binding molecules and nucleic acid sequences encoding such molecules. In particular embodiments, provided herein are human C6 binding molecules (e.g., monoclonal antibodies or antigen binding fragments thereof) with particular light and/or heavy chains variable regions, or light chain and/or heavy chain CDRs, and methods for using such molecules to treat a disease, such as a complement-mediated disease.

The present application claims priority to U.S. Provisional application Ser. No. 63/380,391, filed Oct. 21, 2022, which is herein incorporated by reference in its entirety.

### FIELD OF THE INVENTION

Provided herein are human C6 binding molecules and nucleic acid sequences encoding such molecules. In particular embodiments, provided herein are human C6 binding molecules (e.g., monoclonal antibodies or antigen binding fragments thereof) with particular light and/or heavy chains variable regions, or light chain and/or heavy chain CDRs, and methods for using such molecules to treat a disease, such as a complement-mediated disease.

### BACKGROUND OF THE INVENTION

Complement activation is a key function of the immune system and occurs via three pathways: the classical, alternative and lectin pathways. All pathways result in the cleavage of C5 into C5a and C5b, whereupon the C5b cleavage product binds to C6. Subsequent binding of C7, C8 and multiple C9 molecules leads to assembly of a membrane attack complex on the target cell membrane. However, multiple diseases are caused by excessive membrane attack complex formation and the damage this does to host tissues. A therapeutic approach to inhibit membrane attack complex formation has been to target C5 with an antibody (eculizumab) that prevents its cleavage. Although this has been used successfully to treat complement-mediated disease, an anti-C5 may not be ideal due concomitant suppression of C5a-initiated inflammation, fast turnover of C5 leading to high requirements for antibody and some patients being incompletely responsive or unresponsive to treatment.

### SUMMARY OF THE INVENTION

Provided herein are human C6 binding molecules and nucleic acid sequences encoding such molecules. In particular embodiments, provided herein are human C6 binding molecules (e.g., monoclonal antibodies or antigen binding fragments thereof) with particular light and/or heavy chains variable regions, or light chain and/or heavy chain CDRs, and methods for using such molecules to treat a disease, such as a complement-mediated disease.

In some embodiments, provided herein are compositions comprising a human C6 binding molecule, wherein the human C6 binding molecule comprises: a) a heavy chain variable region, or a first nucleic acid sequence encoding the heavy chain variable region, wherein the heavy chain variable region comprises: i) a CDRH1 amino acid sequence comprising SEQ ID NO: 14, 22, 30, 38, or 46, or any of the preceding with one with one or two conservative amino acid changes, ii) a CDRH2 amino acid sequence comprising SEQ ID NO: 15, 23, 31, 39, or 47, or any of the preceding with one or two conservative amino acid changes, and iii) a CDRH3 amino acid sequence comprising SEQ ID NO: 16, 24, 32, 40, or 48, or any of the preceding with one with one or two conservative amino acid changes, and/or; b) a light chain variable region, or a second nucleic acid sequence encoding the light chain variable region, wherein the light chain variable region comprises: i) a CDRL1 amino acid sequence comprising SEQ ID NO: 18, 26, 34, 42, or 50, or any of the preceding with one with one or two conservative amino acid changes, ii) a CDRL2 amino acid sequence comprising SEQ ID NO: 19, 27, 35, 43, or 51, or any of the preceding with one with one or two conservative amino acid changes, and iii) a CDRL3 amino acid sequence comprising SEQ ID NO: 20, 28, 36, 44, or 52, or any of the preceding with one with one or two conservative amino acid changes. In certain embodiments, the a human C6 binding molecule binds the FIM domain of human C6.

In some embodiments, provided herein are methods of treating or preventing a complement-mediated disease comprising: treating a subject with a human C6 binding molecule as recited above and herein, or an expression vector encoding such human C6 binding molecule, and wherein the subject has, or is suspected to develop, a complement-mediated disease.

In certain embodiments, provide herein are methods of inhibiting Membrane Attack Complex (MAC) formation or activity in a subject comprising: administering to the subject a human C6 binding molecule as recited above and herein, or an expression vector encoding said human C6 binding molecule, in an amount effective to inhibit MAC formation or activity in the subject.

In other embodiments, provided herein are methods of inhibiting of treating, preventing, or reducing symptoms of a disorder mediated by undesired activity of the complement system in a subject comprising: administering to the subject an effective amount of the human C6 binding molecule as recited above and herein, or an expression vector encoding said human C6 binding molecule.

In further embodiments, the complement-mediated disease is selected from the group consisting of: membrane-proliferative glomerulonephritis, cold agglutinin disease, catastrophic antiphospholipid syndrome, antibody-mediated transplantation rejection, ischemia/reperfusion injury, rheumatoid arthritis, atherosclerosis, and Guillain Barre Syndrome. In additional embodiments, the complement-mediated disease is selected from the group consisting of: myasthenia gravis, paroxysmal nocturnal hemoglobinuria, atypical hemolytic uremic syndrome, neuromyelitis optica spectrum disorders, and dense-deposit disease.

In certain embodiments, the human C6 binding molecule is an antibody, minibody, diabody, scFv, or antibody fragment capable of binding human C6. In other embodiments, the antibody fragment is an Fab, or Fv antibody fragment. In some embodiments, the antibody or antibody fragment comprises at least an antigen binding portion of: 1) C601 HFKC4 HuG4K (aka clone 3713) antibody, 2) C601 HFKI HuG4K antibody, 3) C601 HPKI HuG4K antibody, 4) C601 HOKC4 HuG4K antibody, or 5) C601 HPKC4 HuG4K antibody.

In particular embodiments, the heavy chain and/or light chain variable region comprises a human framework region. In additional embodiments, the human C6 binding molecule further comprises a light chain constant region and a CH1 heavy chain constant region. In further embodiments, the human C6 binding molecule further comprises a CH2 heavy chain constant region and/or a CH3 heavy chain constant region. In particular embodiments, the light chain constant region is human or a humanized murine, and/or wherein the CH1, CH2, and CH3 heavy chain constant regions are human or are humanized murine.

In additional embodiments, the human C6 binding molecule is an antibody or antigen binding portion thereof that has an Fc region characterized in that it: i) has an Fc cellular binding site; or ii) has a Fc complement binding site. In other embodiments, the human C6 binding molecule comprises an antibody, wherein the light chain constant region of the antibody is selected from: IgG Kappa and IgG Lambda, and wherein the heavy chain constant region of the antibody is selected from: IgG1, IgG2, IgG3, and IgG4. In other embodiments, the human C6 binding molecule comprises an antibody, or antigen binding portion thereof, which is glycosylated or non-glycosylated. In particular embodiments, the compositions further comprise a physiologically tolerable buffer.

In certain embodiments, the heavy chain variable regions comprises SEQ ID NO: 13, 21, 29, 37, or 45, or any of the preceding with one or more conservative amino acid changes. In additional embodiments, the light chain variable region comprises SEQ ID NO: 17, 25, 33, 41, or 49, or any of the preceding with one or more conservative amino acid changes.

In particular embodiments, provided herein are methods of detecting human C6 in a sample comprising: a) contacting a sample with the human C6 binding molecules above or herein, wherein the sample is suspected of containing human C6, and wherein the human C6 binding molecule forms a complex with the human C6 if present in the sample; and b) detecting the presence or absence of the complex in the sample. In some embodiments, the sample is from a subject that has, or is suspected to develop, complement-mediated disease. In additional embodiments, the human C6 binding molecule comprises a detectable label. In particular embodiments, the methods further comprise contacting the sample with a conjugate molecule capable of binding to the human C6 binding molecule, wherein the conjugate molecule comprises a detectable label.

### DESCRIPTION OF THE FIGURES

FIG. 1 shows a schematic representation of an exemplary IgG molecule with the various regions and sections labeled. The CDRs and framework regions (FR) of one of the two variable region light chains, and one of the two variable region heavy chains, are also labeled.

FIG. 2A shows the nucleotide (SEQ ID NO:1) and amino acid sequence (SEQ ID NO: 2) of 1C9 antibody heavy chain variable region. FIG. 2B shows the nucleotide (SEQ ID NO: 3) and amino acid sequence (SEQ ID NO:4) of 1C9 antibody kappa chain variable region.

FIG. 3A shows the nucleotide (SEQ ID NO:5) and amino acid sequence (SEQ ID NO: 6) of MoC601VH chimeric antibody heavy chain variable region. FIG. 3B shows the nucleotide (SEQ ID NO:7) and amino acid sequence (SEQ ID NO:8) of MoC601VH chimeric antibody kappa chain variable region.

FIG. 4A shows, for humanized antibody variant C601 HFKC4 HuG4k (aka 3713), the: 1) amino acid sequence of the heavy chain variable region (SEQ ID NO:13), including CDRH1 (SEQ ID NO:14), CDRH2 (SEQ ID NO:15), and CDRH3 (SEQ ID NO:16), and 2) amino acid sequence of the kappa (light) chain variable region (SEQ ID NO: 17), including CDRL1 (SEQ ID NO:18), CDRL2 (SEQ ID NO:19), and CDRL3 (SEQ ID NO:20).

FIG. 4B shows, for humanized antibody variant C601 HFKI HuG4k, the: 1) amino acid sequence of the heavy chain variable region (SEQ ID NO:21), including CDRH1 (SEQ ID NO: 22), CDRH2 (SEQ ID NO:23), and CDRH3 (SEQ ID NO:24), and 2) amino acid sequence of the kappa (light) chain variable region (SEQ ID NO:25), including CDRL1 (SEQ ID NO: 26), CDRL2 (SEQ ID NO:27), and CDRL3 (SEQ ID NO:28).

FIG. 4C shows, for humanized antibody variant C601 HPKI HuG4k, the: 1) amino acid sequence of the heavy chain variable region (SEQ ID NO:29), including CDRH1 (SEQ ID NO: 30), CDRH2 (SEQ ID NO:31), and CDRH3 (SEQ ID NO:32), and 2) amino acid sequence of the kappa (light) chain variable region (SEQ ID NO:33), including CDRL1 (SEQ ID NO: 34), CDRL2 (SEQ ID NO:35), and CDRL3 (SEQ ID NO:36).

FIG. 4D shows, for humanized antibody variant C601 HOKC4 HuG4k, the: 1) amino acid sequence of the heavy chain variable region (SEQ ID NO:37), including CDRH1 (SEQ ID NO:38), CDRH2 (SEQ ID NO:39), and CDRH3 (SEQ ID NO:40), and 2) amino acid sequence of the kappa (light) chain variable region (SEQ ID NO:41), including CDRL1 (SEQ ID NO:42), CDRL2 (SEQ ID NO:43), and CDRL3 (SEQ ID NO:44).

FIG. 4E shows, for humanized antibody variant C601 HPKC4 HuG4k, the: 1) amino acid sequence of the heavy chain variable region (SEQ ID NO:45), including CDRH1 (SEQ ID NO:46), CDRH2 (SEQ ID NO:47), and CDRH3 (SEQ ID NO:48), and 2) amino acid sequence of the kappa (light) chain variable region (SEQ ID NO:49), including CDRL1 (SEQ ID NO:50), CDRL2 (SEQ ID NO:51), and CDRL3 (SEQ ID NO:52).

FIG. 5 shows the kinetics parameters for the binding of C6 to the chimeric and five lead humanized C601 antibodies. The kinetics of C6 binding was compared for the purified chimeric and lead humanized C601 using surface plasmon resonance (SPR, Biacore 8K). IgG were captured on a protein G sensor chip and binding of C6 measured using a three-fold concentration series of C6 from 0.03 nM-25 nM.

FIG. 6 shows the same kinetic parameters for binding of C6 to the chimeric and five lead humanized C601 antibodies, this time using a two-fold concentration series from 0.31 nM-20 nM C6 was used.

FIG. 7 shows results of solubility determinations for the five lead humanized C601 antibodies using solvent absorption concentrators (MWCO 7500 Da).

FIG. 8 shows results for a serum stability assessment of the chimeric and five lead humanized C601 candidate antibodies.

FIG. 9 shows recombinant chimeric 1C9 were produced and tested in a hemolytic assay using human (NHS), rat or guinea pig (GP) serum as the source of complement. C19 inhibits complement-mediated hemolysis in all these assays, suggesting that it cross-reacts with rat and guinea pig C6.

FIG. 10: Binding of 1C9 to different truncated C6 proteins (10A) were detected by ELISA, suggesting that 1C9 binds to the FIM1-2 domains of C6 (10B), which was confirmed by SPR using purified FIM1-2 and 1C9 (10C).

FIG. 11: Clone 3713 and its controls were tested in inhibiting complement-mediated hemolysis in human (A), NHP (B) and rat (C) complement systems. Clone 3713 also protected PNH RBCs from hemolysis in a modified Ham's test (D).

FIG. 12. WT rats infused with human RBCs were treated with X μM of clone 3713 or hIgG by i.v. injections. 20 min later, blood (A) and urine (B) samples were collected, and hemolysis were assessed. A representative picture of the urine samples is shown (C).

### DEFINITIONS

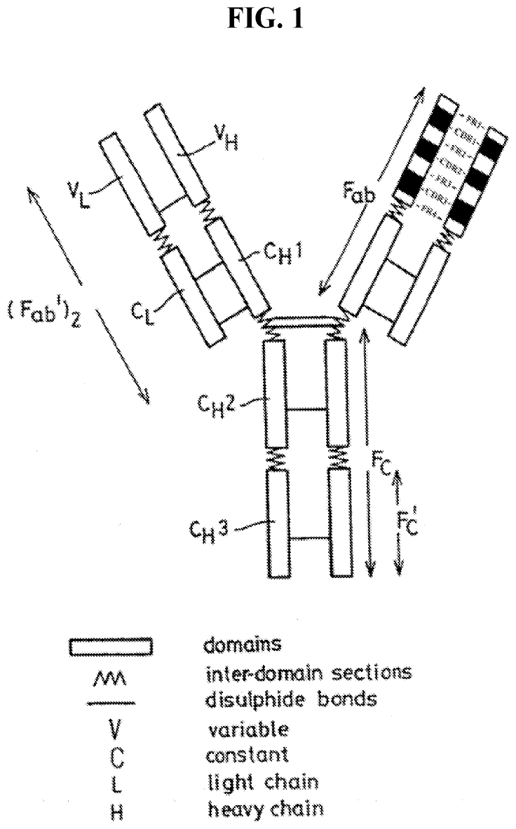

To facilitate an understanding of the invention, a number of terms are defined below. The term “antibody,” as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each variable region (VH or VL) contains 3 CDRs, designated CDR1, CDR2 and CDR3 (see, FIG. 1). Each variable region also contains 4 framework sub-regions, designated FR1, FR2, FR3 and FR4 (see, FIG. 1), which may be human framework sub-regions.

As used herein, the term “antibody fragment or portion” refers to a portion of an intact antibody. Examples of antibody fragments or portions include, but are not limited to, linear antibodies, single-chain antibody molecules, Fv, Fab and F(ab′) 2 fragments, and multi-specific antibodies formed from antibody fragments. The antibody fragments preferably retain at least part of the heavy and/or light chain variable region.

As used herein, the terms “complementarity determining region” and “CDR” refer to the regions that are primarily responsible for antigen-binding. There are three CDRs in a light chain variable region (CDRL1, CDRL2, and CDRL3), and three CDRs in a heavy chain variable region (CDRH1, CDRH2, and CDRH3).

As used herein, the term “framework” refers to the residues of the variable region other than the CDR residues. There are four separate framework sub-regions that make up the framework: FR1, FR2, FR3, and FR4 (see, FIG. 1). In order to indicate if the framework sub-region is in the light or heavy chain variable region, an “L” or “H” may be added to the sub-region abbreviation (e.g., “FRL1” indicates framework sub-region 1 of the light chain variable region). It is noted that, in certain embodiments, the human C6 binding molecules of the present invention may have less than a complete framework (e.g. the human C6 binding molecule may have a portion of a framework that only contains one or more of the four sub-regions).

As used herein, the term “fully human framework” means a framework with an amino acid sequence found naturally in humans. Examples of fully human frameworks, include, but are not limited to, KOL, NEWM, REI, EU, TUR, TEI, LAY and POM (See, e.g., Kabat et al., (1991) Sequences of Proteins of Immunological Interest, US Department of Health and Human Services, NIH, USA; and Wu et al., (1970) J. Exp. Med. 132, 211-250, both of which are herein incorporated by reference). In certain embodiments, the human C6 binding molecules herein have a fully human framework.

As used herein, the terms “subject” and “patient” refer to any animal, such as a mammal like a dog, cat, bird, livestock, and preferably a human.

As used herein, the term “codon” or “triplet” refers to a group of three adjacent nucleotides which specify one of the naturally occurring amino acids found in polypeptides. The term also includes codons which do not specify any amino acid. It is also noted that, due to the degeneracy of the genetic code, there are many codons that code for the same amino acid. As such, many of the bases of the nucleic acid sequences of the present invention can be changed without changing the actual amino acid sequence that is encoded. The present disclosure is intended to encompass all such nucleic acid sequences.

As used herein, the terms “an oligonucleotide having a nucleotide sequence encoding a polypeptide,” “polynucleotide having a nucleotide sequence encoding a polypeptide,” and “nucleic acid sequence encoding a peptide” means a nucleic acid sequence comprising the coding region of a particular polypeptide. The coding region may be present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc., or a combination of both endogenous and exogenous control elements.

Also, as used herein, there is no size limit or size distinction between the terms “oligonucleotide” and “polynucleotide.” Both terms simply refer to molecules composed of nucleotides. Likewise, there is no size distinction between the terms “peptide” and “polypeptide.” Both terms simply refer to molecules composed of amino acid residues.

As used herein, the term “the complement of” a given sequence is used in reference to the sequence that is completely complementary to the sequence over its entire length. For example, the sequence 5′-A-G-T-A-3′ is “the complement” of the sequence 3′-T-C-A-T-5′.

The term “isolated” when used in relation to a nucleic acid, as in “an isolated oligonucleotide” or “isolated polynucleotide” or “isolated nucleic acid sequence encoding an C6 binding molecule” refers to a nucleic acid sequence that is identified and separated from at least one contaminant nucleic acid with which it is ordinarily associated (e.g. host cell proteins).

As used herein, the term “purified” or “to purify” refers to the removal of contaminants from a sample. For example, C6 binding molecules (e.g., antibodies or antibody fragments) may be purified by removal of contaminating non-immunoglobulin proteins; they are also purified by the removal of immunoglobulins that do not bind to the same antigen. The removal of non-immunoglobulin proteins and/or the removal of immunoglobulins that do not bind the particular antigen results in an increase in the percentage of antigen specific immunoglobulins in the sample. In another example, recombinant antigen-specific polypeptides are expressed in bacterial host cells and the polypeptides are purified by the removal of host cell proteins; the percentage of recombinant antigen-specific polypeptides is thereby increased in the sample.

As used herein, the term “Fc region” refers to a C-terminal region of an immunoglobulin heavy chain. The “Fc region” may be a native sequence Fc region or a variant Fc region (e.g., with increased or decreased effector functions).

### DESCRIPTION OF THE INVENTION

Provided herein are human C6 binding molecules and nucleic acid sequences encoding such molecules. In particular embodiments, provided herein are human C6 binding molecules (e.g., monoclonal antibodies or antigen binding fragments thereof) with particular light and/or heavy chains variable regions, or light chain and/or heavy chain CDRs, and methods for using such molecules to treat a disease, such as a complement-mediated disease.

In certain embodiments, the human C6 binding molecules are used to treat disease. For example, provided herein are methods of treating or preventing a complement-mediated disease comprising: treating a subject with a human C6 binding molecule as recited above and herein, or an expression vector encoding such human C6 binding molecule, and wherein the subject has, or is suspected to develop, a complement-mediated disease. In further embodiments, the complement-mediated disease is selected from the group consisting of: membrane-proliferative glomerulonephritis, cold agglutinin disease, catastrophic antiphospholipid syndrome, antibody-mediated transplantation rejection, ischemia/reperfusion injury, rheumatoid arthritis, atherosclerosis, and Guillain Barre Syndrome. In additional embodiments, the complement-mediated disease is selected from the group consisting of: myasthenia gravis, paroxysmal nocturnal hemoglobinuria, atypical hemolytic uremic syndrome, neuromyelitis optica spectrum disorders, and dense-deposit disease.

In certain embodiments, provided herein are methods of using a human C6 binding molecule described herein to inhibit Membrane Attack Complex (MAC) formation or activity in a subject (e.g., human), the method comprising administering to the subject such human C6 binding molecules in an amount effective to inhibit MAC formation or activity in the subject.

In other embodiments, provided herein are methods of regenerating nerves, or treating damaged or degenerated nerves, or reducing or delaying degeneration of nerves, in a subject (e.g., human), comprising administering to the subject a therapeutically effective amount of the human C6 binding molecules described herein. In some embodiments, the subject is suffering from a physical injury of the nerves, such as a traumatic injury (e.g., from an accident), a surgical injury or non-traumatic injury (e.g., a nerve compression). In certain embodiments, administration is at or near the site of injury. In some embodiments, the subject is suffering from an immune-mediated inflammatory disorder or progressive neurodegenerative disorder, or a chronic demyelinating neuropathy, such as multiple sclerosis (MS), or a neurodegenerative disorder, such as myasthenia gravis or amyotrophic lateral sclerosis (ALS).

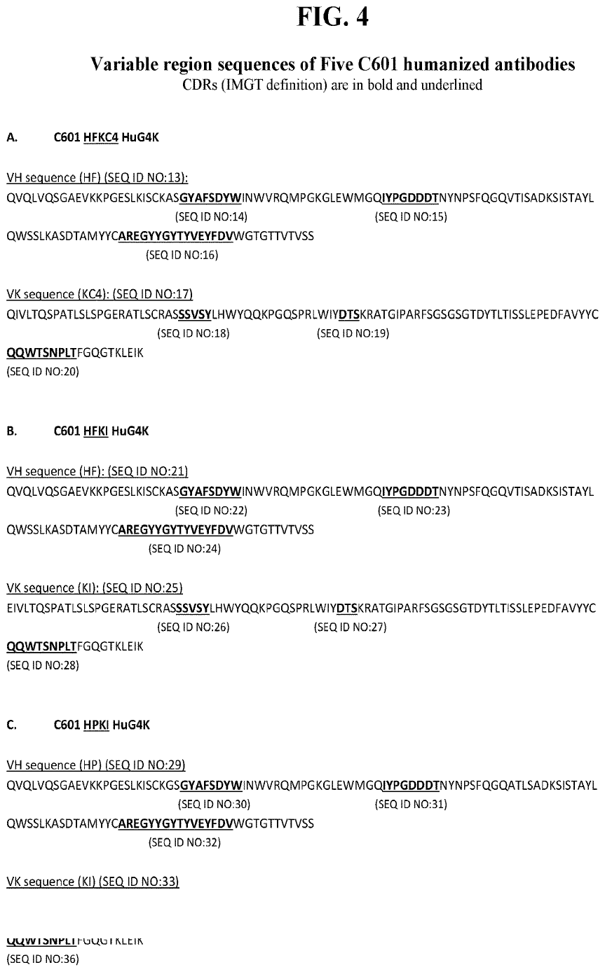

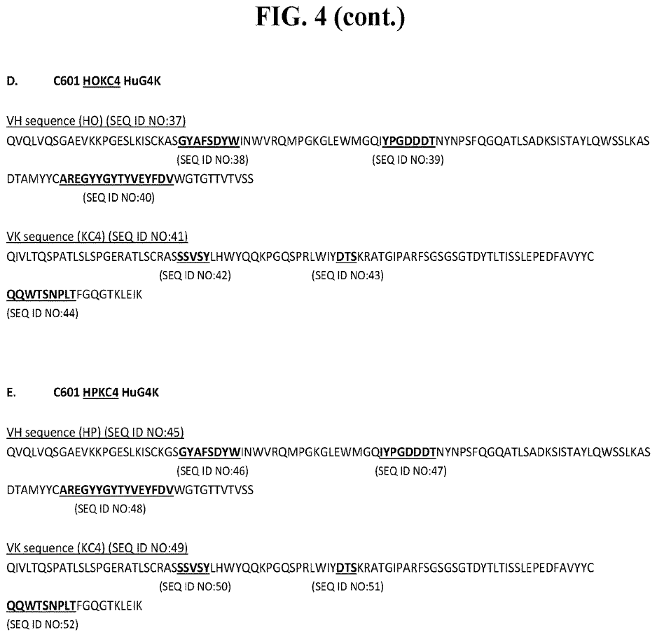

In certain embodiments, the human C6 binding molecules comprise one or more of the variable regions or CDRs shown in SEQ ID NOS: 13-52 and/or variable regions or CDRs with one or more conservative or non-conservative amino acid changes in these SEQ ID NOS: 13-52. Also provided are nucleic acid sequences substantially similar to SEQ ID NOS: 13-52 (e.g., sequences with at least 80 . . . 90 . . . 95% . . . or 99% sequence identity). Changes to the amino acid sequences of the CDRs or variable regions (see FIG. 4) may be generated by changing the nucleic acid sequence encoding the amino acid sequence. A nucleic acid sequence encoding a variant of a given CDR or variable region may be prepared by methods known in the art using the guidance of the present specification for particular sequences. These methods include, but are not limited to, preparation by site-directed (or oligonucleotide-mediated) mutagenesis, PCR mutagenesis, and cassette mutagenesis of an earlier prepared nucleic acid encoding the CDR or variable region.

Briefly, in carrying out site-directed mutagenesis of DNA, the starting DNA is altered by first hybridizing an oligonucleotide encoding the desired mutation to a single strand of such starting DNA. After hybridization, a DNA polymerase is used to synthesize an entire second strand, using the hybridized oligonucleotide as a primer, and using the single strand of the starting DNA as a template. Thus, the oligonucleotide encoding the desired mutation is incorporated in the resulting double-stranded DNA.

PCR mutagenesis is also suitable for making amino acid sequence variants of the starting CDR (see, e.g., Vallette et. al., (1989) Nucleic Acids Res. 17:723-733, hereby incorporated by reference). Briefly, when small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template.

Another method for preparing variants, cassette mutagenesis, is based on the technique described by Wells et al., (1985) Gene 34:315-323, hereby incorporated by reference. The starting material is the plasmid (or other vector) comprising the starting CDR or variant region DNA to be mutated. The codon(s) in the starting DNA to be mutated are identified. There should be a unique restriction endonuclease site on each side of the identified mutation site(s). If no such restriction sites exist, they may be generated using the above-described oligonucleotide-mediated mutagenesis method to introduce them at appropriate locations in the starting polypeptide DNA. The plasmid DNA is cut at these sites to linearize it. A double-stranded oligonucleotide encoding the sequence of the DNA between the restriction sites but containing the desired mutation(s) is synthesized using standard procedures, wherein the two strands of the oligonucleotide are synthesized separately and then hybridized together using standard techniques. This double-stranded oligonucleotide is referred to as the cassette. This cassette is designed to have 5′ and 3′ ends that are compatible with the ends of the linearized plasmid, such that it can be directly ligated to the plasmid. This plasmid now contains the mutated DNA sequence.

Alternatively, or additionally, the desired amino acid sequence encoding a CDR variant, or variable region variant, can be determined, and a nucleic acid sequence encoding such amino acid sequence variant can be generated synthetically. Conservative modifications in the amino acid sequences of the CDRs or variable region may also be made. Naturally occurring residues are divided into classes based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.
   Conservative substitutions will entail exchanging a member of one of these classes for another member of the same class in a particular variable region or CDR, such as in SEQ ID NOS: 13-52.

The CDRs of the present invention may be employed with any type of suitable framework. In some embodiments, the CDRs are used with fully human frameworks, or framework sub-regions. For example, the NCBI web site contains the sequences for known human framework regions. Examples of human VH sequences include, but are not limited to, VH1-18, VH1-2, VH1-24, VH1-3, VH1-45, VH1-46, VH1-58, VH1-69, VH1-8, VH2-26, VH2-5, VH2-70, VH3-11, VH3-13, VH3-15, VH3-16, VH3-20, VH3-21, VH3-23, VH3-30, VH3-33, VH3-35, VH3-38, VH3-43, VH3-48, VH3-49, VH3-53, VH3-64, VH3-66, VH3-7, VH3-72, VH3-73, VH3-74, VH3-9, VH4-28, VH4-31, VH4-34, VH4-39, VH4-4, VH4-59, VH4-61, VH5-51, VH6-1, and VH7-81, which are provided in Matsuda et al., (1998) J. Exp. Med. 188:1973-1975, that includes the complete nucleotide sequence of the human immunoglobulin chain variable region locus, herein incorporated by reference. Examples of human VK sequences include, but are not limited to, A1, A10, All, A14, A17, A18, A19, A2, A20, A23, A26, A27, A3, A30, A5, A7, B2, B3, L1, L10, L11, L12, L14, L15, L16, L18, L19, L2, L20, L22, L23, L24, L25, L4/18a, L5, L6, L8, L9, O1, O11, O12, O14, O18, O2, O4, and O8, which are provided in Kawasaki et al., (2001) Eur. J. Immunol. 31:1017-1028; Schable and Zachau, (1993) Biol. Chem. Hoppe Seyler 374:1001-1022; and Brensing-Kuppers et al., (1997) Gene 191:173-181, all of which are herein incorporated by reference. Examples of human VL sequences include, but are not limited to, V1-11, V1-13, V1-16, V1-17, V1-18, V1-19, V1-2, V1-20, V1-22, V1-3, V1-4, V1-5, V1-7, V1-9, V2-1, V2-11, V2-13, V2-14, V2-15, V2-17, V2-19, V2-6, V2-7, V2-8, V3-2, V3-3, V3-4, V4-1, V4-2, V4- 3, V4-4, V4-6, V5-1, V5-2, V5-4, and V5-6, which are provided in Kawasaki et al., (1997) Genome Res. 7:250-261, herein incorporated by reference. Fully human frameworks can be selected from any of these functional germline genes. Generally, these frameworks differ from each other by a limited number of amino acid changes. These frameworks may be used with the CDRs described herein. Additional examples of human frameworks which may be used with the CDRs of the present invention include, but are not limited to, KOL, NEWM, REI, EU, TUR, TEI, LAY and POM (See, e.g., Kabat et al., (1991) Sequences of Proteins of Immunological Interest, US Department of Health and Human Services, NIH, USA; and Wu et al., (1970), J. Exp. Med. 132:211-250, both of which are herein incorporated by reference).

In certain embodiments, the human C6 binding molecules of the present invention comprise antibodies or antibody fragments (e.g., comprising one or more of the CDRs described herein, such as in FIG. 4). An antibody, or antibody fragment, of the present invention can be prepared, for example, by recombinant expression of immunoglobulin light and heavy chain genes in a host cell. For example, to express an antibody recombinantly, a host cell may be transfected with one or more recombinant expression vectors carrying DNA fragments encoding the immunoglobulin light and heavy chains of the antibody such that the light and heavy chains are expressed in the host cell and, preferably, secreted into the medium in which the host cell is cultured, from which medium the antibody can be recovered. Standard recombinant DNA methodologies may be used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al., all of which are herein incorporated by reference.

In certain antibodies, the anti-C6 antibodies, or fragments, thereof prepared herein have an IgG isotype constant regions as shown in Table 1 below.

TABLE 1

| IgG Isotype | |
|---|---|
| Constant region | Accession # |
| IgG Light Kappa | P0DOX7.1 |
| IgG Light Lambda | P0DOX8.1 |
| IgG1 Heavy | P01857 |
| IgG2 Heavy | P01859 |
| IgG3 Heavy | P01860 |
| IgG4 Heavy | P01861 |

To express an antibody with one or more of the CDRs herein, DNA fragments encoding the light and heavy chain variable regions are first obtained. These DNAs can be obtained by amplification and modification of germline light and heavy chain variable sequences using the polymerase chain reaction (PCR).

Once the germline VH and VL fragments are obtained, these sequences can be mutated to encode one or more of the CDR amino acid sequences disclosed herein (see, FIG. 4). The amino acid sequences encoded by the germline VH and VL DNA sequences may be compared to the CDRs sequence(s) desired to identify amino acid residues that differ from the germline sequences. Then the appropriate nucleotides of the germline DNA sequences are mutated such that the mutated germline sequence encodes the selected CDRs, using the genetic code to determine which nucleotide changes should be made. Mutagenesis of the germline sequences may be carried out by standard methods, such as PCR-mediated mutagenesis (in which the mutated nucleotides are incorporated into the PCR primers such that the PCR product contains the mutations) or site-directed mutagenesis. In other embodiments, the variable region is synthesized de novo (e.g., using a nucleic acid synthesizer).

Once DNA fragments encoding the desired VH and VL segments are obtained (e.g., by amplification and mutagenesis of germline VH and VL genes, or synthetic synthesis, as described above), these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operably linked to another DNA fragment encoding another polypeptide, such as an antibody constant region or a flexible linker. The isolated DNA encoding the VH region can be converted to a full-length heavy chain gene by operably linking the VH-encoding DNA to another DNA molecule encoding heavy chain constant regions (CH1, CH2 and CH3). The sequences of mouse and human heavy chain constant region genes are known in the art and DNA fragments encompassing these regions can be obtained by standard PCR amplification. The heavy chain constant region can be, for example, an IgG1, IgG2, IgG3, IgG4, IgA, IgE, IgM or IgD constant region. For a Fab fragment heavy chain gene, the VH-encoding DNA can be operably linked to another DNA molecule encoding only the heavy chain CH1 constant region.

The isolated DNA encoding the VL region can be converted to a full-length light chain gene (as well as a Fab light chain gene) by operably linking the VL-encoding DNA to another DNA molecule encoding the light chain constant region, CL. The sequences of mouse and human light chain constant region genes are known in the art (see e.g., Kabat, E. A., et al., (1991) Sequences of Proteins of immunological Interest, Fifth Edition, U.S. Department of Health and Human Services. NIH Publication No. 91-3242) and DNA fragments encompassing these regions can be obtained by standard PCR amplification. The light chain constant region can be a kappa or lambda constant region.

To create a scFv gene, the VH- and VL-encoding DNA fragments may be operably linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see e.g., Huston et al., (1988) Proc. Natl. Acad. Sci. USA 85:5879-5883; and McCafferty et al., (1990) Nature 348:552-554), all of which are herein incorporated by reference).

To express the antibodies, or antibody fragments of the invention, DNAs encoding partial or full-length light and heavy chains, (e.g. obtained as described above), may be inserted into expression vectors such that the genes are operably linked to transcriptional and translational control sequences. In this context, the term “operably linked” is intended to mean that an antibody gene is ligated into a vector such that transcriptional and translational control sequences within the vector serve their intended function of regulating the transcription and translation of the antibody gene. The expression vector and expression control sequences are generally chosen to be compatible with the expression host cell used. The antibody light chain gene and the antibody heavy chain gene can be inserted into separate vectors or, more typically, both genes are inserted into the same expression vector. The antibody genes may be inserted into the expression vector by standard methods (e.g., ligation of complementary restriction sites on the antibody gene fragment and vector, or blunt end ligation if no restriction sites are present). Prior to insertion of the light or heavy chain sequences, the expression vector may already carry antibody constant region sequences. For example, one approach to converting the VH and VL sequences to full-length antibody genes is to insert them into expression vectors already encoding heavy chain constant and light chain constant regions, respectively, such that the VH segment is operably linked to the CH segment(s) within the vector and the VL segment is operably linked to the CL segment within the vector. Additionally, or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the disclosure may carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990), herein incorporated by reference. It will be appreciated by those skilled in the art that the design of the expression vector, including the selection of regulatory sequences may depend on such factors as the choice of the host cell to be transformed, the level of expression of protein desired, etc. In certain embodiments, regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma virus. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al., all of which are herein incorporated by reference.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neomycin gene (for G418 selection).

For expression of the light and heavy chains, the expression vector(s) encoding the heavy and light chains may be transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like.

In certain embodiments, the expression vector used to express the human C6 binding molecules of the present invention are viral vectors, such as retro-viral vectors. Such viral vectors may be employed to generate stably transduced cell lines (e.g. for a continues source of the C6 binding molecules). In some embodiments, the GPEX gene product expression technology (from Catalent, Somerset, NJ) is employed to generate C6 binding molecules (and stable cell lines expressing the C6 binding molecules). In particular embodiments, the expression technology described in WO0202783 and WO0202738 to Bleck et al. (both of which are herein incorporated by reference in their entireties) is employed.

Mammalian host cells for expressing the recombinant antibodies of the invention include, for example, PER.C6™ cells (Crucell, The Netherlands), Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. In other embodiments, the host cells express GnT III as described in WO9954342 and U.S. Pat. Pub. 20030003097, both herein incorporated by reference, such that expressed C6 binding molecules have increased ADCC activity. When recombinant expression vectors encoding antibody genes are introduced into mammalian host cells, the antibodies are generally produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. It will be understood that variations on the above procedure are within the scope of the present disclosure. For example, it may be desirable to transfect a host cell with DNA encoding either the light chain or the heavy chain of an antibody of this disclosure. Recombinant DNA technology may also be used to remove some or all of the DNA encoding either or both of the light and heavy chains that is not necessary for binding to C6. The molecules expressed from such truncated DNA molecules are also encompassed by the antibodies of the invention. In addition, bi-functional antibodies may be produced in which one heavy and one light chain are an antibody of the invention and the other heavy and light chain are specific for an antigen other than C6 (e.g., by crosslinking an antibody of the invention to a second antibody by standard chemical crosslinking methods).

In certain embodiments, the antibodies and antibody fragments of the present invention are produced in transgenic animals. For example, transgenic sheep and cows may be engineered to produce the antibodies or antibody fragments in their milk (see, e.g., Pollock D P, et al., (1999) Transgenic milk as a method for the production of recombinant antibodies. J. Immunol. Methods 231:147-157, herein incorporated by reference). The antibodies and antibody fragments of the present invention may also be produced in plants (see, e.g., Larrick et al., (2001) Production of secretory IgA antibodies in plants. Biomol. Eng. 18:87-94, herein incorporated by reference). Additional methodologies and purification protocols are provided in Humphreys et al., (2001) Therapeutic antibody production technologies: molecules applications, expression and purification, Curr. Opin. Drug Discov. Devel. 4:172-185, herein incorporated by reference. In certain embodiments, the antibodies or antibody fragments of the present invention are produced by transgenic chickens (see, e.g., US Pat. Pub. Nos. 20020108132 and 20020028488, both of which are herein incorporated by reference).

In certain embodiments, the human C6 binding molecules of the present invention (e.g., as antibodies or antibody fragments) are useful for immunoassays which detect or quantify human C6 in a sample (e.g., a purified blood sample from a subject). In some embodiments, an immunoassay for C6 typically comprises incubating a biological sample in the presence of a detectably labeled antibody or antibody fragment of the present invention capable of selectively binding to C6, and detecting the labeled peptide or antibody which is bound in a sample. Various clinical assay procedures are well known in the art.

The present disclosure provides immunoassay methods for determining the presence, amount or concentration of human C6 in a test sample. Any suitable assay known in the art can be used in such a method. Examples of such assays include, but are not limited to, immunoassay, such as sandwich immunoassay (e.g., monoclonal-polyclonal sandwich immunoassays, including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, Minn.)), competitive inhibition immunoassay (e.g., forward and reverse), fluorescence polarization immunoassay (FPIA), enzyme multiplied immunoassay technique (EMIT), an ARCHITECT assay (ABBOTT), a bioluminescence resonance energy transfer (BRET), and homogeneous chemiluminescent assay, etc.

A human C6 binding molecule can be captured on beads or nitrocellulose, or on any other solid support which is capable of immobilizing soluble proteins (e.g., magnetic beads). A human C6 containing sample is then added to the support which is subsequently washed with suitable buffers to remove unbound proteins. A second, detectably labeled, molecule (e.g., antibody or peptide) that can bind to the human C6 binding molecule is added to the solid phase support that can then be washed with the buffer a second time to remove unbound molecules. The amount of bound label on the solid support can then be detected by known methods.

Detectably labeling the human C6 binding molecule can be accomplished by coupling to an enzyme for use in an enzyme immunoassay (EIA), or enzyme-linked immunosorbent assay (ELISA). The linked enzyme reacts with the exposed substrate to generate a chemical moiety which can be detected, for example, by spectrophotometric, fluorometric or by visual means. Enzymes which can be used to detectably label the human C6 binding molecules of the present invention include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

In some embodiments of the present invention, human C6 which is detected by the above assays can be present in a biological sample. Any sample containing human C6 can be used. Preferably, the sample is a biological fluid such as, for example, blood, serum, lymph, urine, cerebrospinal fluid, amniotic fluid, synovial fluid, a tissue extract or homogenate, and the like. However, the invention is not limited to assays using only these samples, as it is possible for one of ordinary skill in the art to determine suitable conditions which allow the use of other samples.

In situ detection can be accomplished by removing a histological specimen from a patient and providing the combination of labeled human C6 binding molecules of the present disclosure to such a specimen. The human C6 binding molecule is preferably provided by applying or by overlaying the labeled C6 binding molecule to a biological sample. Through the use of such a procedure, it is possible to determine not only the presence of C6, but also the distribution of C6 in the examined tissue.

In certain embodiments, provided here are kits for the detection of C6 that include a human C6 detection molecule. Such kits may include any of the immunodiagnostic reagents described herein and may further include instructions for the use of the immunodiagnostic reagents in immunoassays for determining the presence of human C6 in a test sample. The kits may also include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit may be provided in suitable containers as necessary, e.g., a microtiter plate. The kit can further include containers for holding or storing a sample (e.g., a container or cartridge for a sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

### EXAMPLES

The following examples are provided in order to demonstrate and further illustrate certain preferred embodiments and aspects of the present invention and are not to be construed as limiting the scope thereof.

### Example 1

### Sequence Determination of the 1C9 (MoC601) Antibody

This example describes determining the nucleic acid and amino acid sequences for the 1C9 mouse anti-C6 monoclonal antibody.

RNA Preparation from Hybridoma Cells.

Cryopreserved hybridoma cells 1C9 were supplied by Cleveland Clinic and were grown in RPMI-1640 supplemented with 20% Ultra low IgG fetal bovine serum (Gibco) to provide cells for RNA isolation. Two cell pellets (each 5×10⁶ cells) were washed in PBS and stored at −80 C prior to processing. RNA was isolated from each pellet using the Qiagen RNeasy Kit, following the manufacturer's protocol.

First Strand cDNA Synthesis

A sample (4 μg) of one of the two 1C9 RNA preparations and two 4 samples of the second RNA preparation were each reverse-transcribed to produce cDNA using the Cytiva First strand cDNA synthesis kit, following the manufacturer's protocol, and purified. Thus, three independent cDNA products were generated in order to detect any cDNA mutations induced by the reverse transcriptase.

cDNA Sequence Determination for 1C9 (MoC601) Antibody

Using an IsoStrip™ Mouse Monoclonal Antibody Isotyping Kit (Roche) and 1C9 hybridoma cell culture supernatant, the isotype of the 1C9 antibody was determined to be mouse IgG1K. Therefore, cDNA samples were amplified by PCR using each kappa light chain leader sequence primer (MKV1-11) with MKC, and each heavy chain leader sequence primer (MHV1-14) with MHCG1, using the Phusion Flash High-Fidelity PCR Master Mix.

Where primer combinations gave amplification products of the correct sizes, these were purified using the QIAquick PCR purification kit, and sequenced at Genewiz (Azenta Life Sciences) in both directions using the M13-Forward and M13-Reverse primers. For the VH, reactions with MHV7 and MHCG1 from each of the three independent cDNA products gave the sequence of a functional VH. For the VK, the reactions with MKV5 and MKC all gave sequence of the same functional VK so, as for the VH, three independent sets of sequence information were obtained.

### 1C9 VH and VK DNA Sequences

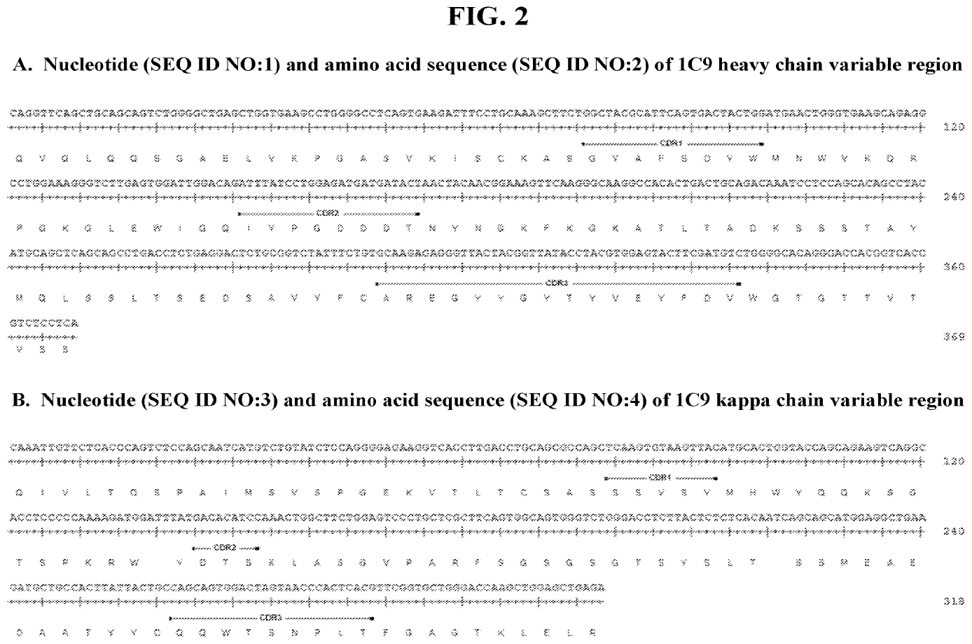

The nucleotide and amino acid sequences of the 1C9 VH and VK PCR products are shown in FIG. 2.

Germline analysis of the 1C9 variable region sequences shows that the VH has a very high identity (98.0%) to the mouse germline V segment IGHV1-80*01, with only 2 somatic mutations, one in each of CDRs 1 and 2. Framework 4 is encoded by IGHJ1*03 and is unmutated. CDR3 is 16 residues, which easily puts 1C9 VH in the top 10% of mouse VH regions in terms of CDR3 length. The VK shows highest identity (96.8%) to the mouse germline V segment IGKV4-59*01. CDRs 1 and 2 are unmutated but there are 2 somatic mutations within framework 1. Framework 4 is encoded by IGKJ5*01 and has a mutation at its final residue.

1C9 variable region sequences were analysed for sequence liabilities. The variable regions do not contain any unusual proline, methionine or cysteine residues as, where these residues occur, they are all encoded by the mouse germline genes. There are no N-linked glycosylation sites but there are four aspartate isomerisation risk motifs and an asparagine deamidation risk motif. When these risks are within the frameworks, they can be eliminated by the choice of human frameworks during the humanisation design process. However, there is a double DD aspartate isomerisation risk motif within VH CDR2: DDD. Unfortunately, the second D residue is a somatic mutation and, as such, may be important for antigen binding. The risk of isomerisation may need to be mitigated by formulation.

### Example 2

### Sequence Determination and Characterization of MoC601 Chimeric Antibody

This example describes determining the nucleic acid and amino acid sequences for the MoC601 Chimeric anti-C6 monoclonal antibody and its characterization.

### Construction of the Chimeric C601 Expression Vectors

Construction of chimeric expression vectors entailed using ligase-independent cloning (LIC) to insert mouse C601 variable regions into vectors that contained DNA encoding the human IgG4 constant region, the human kappa constant region or the CH1 of the human IgG1 constant region only (to enable production of a Fab fragment). The amino acid sequences of these human constant regions are given in Table 2.

TABLE 2
Amino acid sequences of signal peptide and constant regions

| Name | Sequence |
|---|---|
| Human | MEAPAQLLFLLLLWLPDTTG (SEQ ID NO: 9) |
| kappa chain | |
| signal | |
| Human | ASTKGPSVFPLAPCSRSTSESTAALGCLVKDYFPEPVTVSWNSGALTSGVHTFPAVLQ |
| IgG4 CH1-CH3 | SSGLYSLSSVVTVPSSSLGTKTYTCNVDHKPSNTKVDKRVESKYGPPCPPCPAPEFLGGPSVFL |
| | FPPKPKDTLMISRTPEVTCVVVDVSQEDPEVQFNWYVDGVEVHNAKTKPREEQFNSTYRVVSVL |
| | TVLHQDWLNGKEYKCKVSNKGLPSSIEKTISKAKGQPREPQVYTLPPSQEEMTKNQVSLTCLVK |
| | GFYPSDIAVEWESNGQPENNYKTTPPVLDSDGSFFLYSRLTVDKSRWQEGNVFSCSVMHEALHN |
| | HYTQKSLSLSLGK (SEQ ID NO: 10) |
| Human | RTVAAPSVFIFPPSDEQLKSGTASVVCLLNNFYPREAKVQWKVDNALQSGNSQESVTE |
| kappa | QDSKDSTYSLSSTLTLSKADYEKHKVYACEVTHQGLSSPVTKSFNRGEC (SEQ ID NO: 11) |
| Human | ASTKGPSVFPLAPSSKSTSGGTAALGCLVKDYFPEPVTVSWNSGALTSGVHTFPAVLQ |
| IgG1 CH1 | SSGLYSLSSVVTVPSSSLGTQTYICNVNHKPSNTKVDKKVEPKSC (SEQ ID NO: 12) |
| (for Fab) | |

*The human kappa chain signal peptide is used in expression of all chains, heavy and kappa.

The vectors (pCMV modified) were digested with BfuA1 (BspM1) and then compatible overhangs were generated with T4 DNA polymerase 3′-5′ exonuclease activity in presence of dATP.

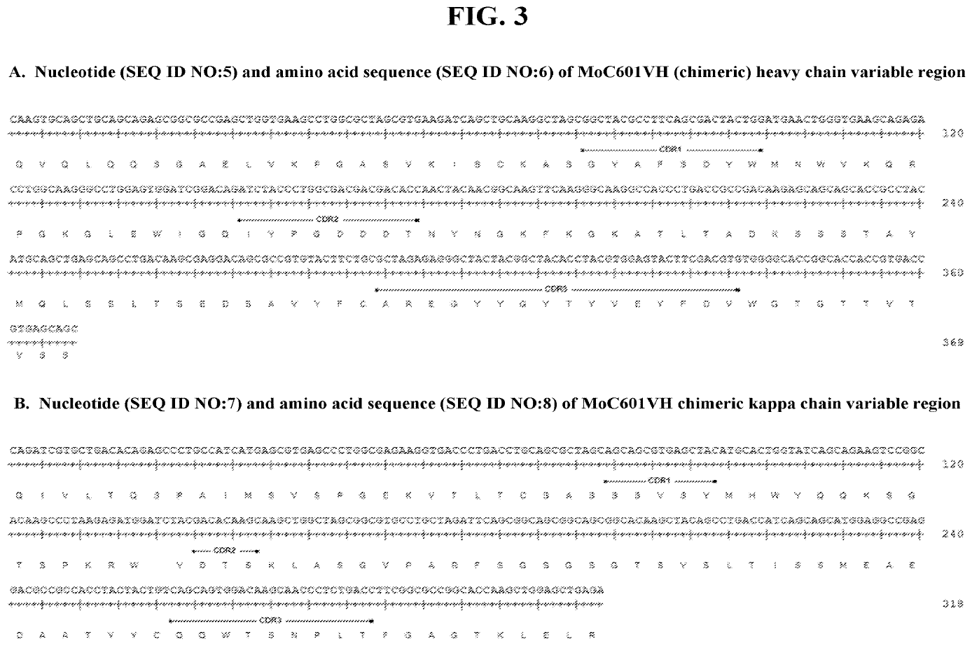

Using software algorithms at Genewiz (Azenta Life Sciences), the nucleotide sequences for MoC601 VH and VK were optimized to use codons preferentially utilized by CHO cells. Gene fragments for MoC601 VH and VK were then synthesized by Genewiz to include the 3′ end of the leader sequence (most of the leader sequence is present in the vector) and beginning of the constant region. The variable region sequences that are used in the generation of the chimeric molecules are named MoC601VH and MoC601VK and are shown in FIG. 3. Complementary overhangs to those in the vectors were generated in the gene fragments by T4 DNA polymerase treatment in presence of dTTP. Vector and inserts were incubated at room temperature and transformed into chemically-competent TOP10 bacteria, which were plated on kanamycin plates. Several colonies were screened by PCR using primer ‘HCMVipro for’ and the appropriate constant region reverse primer. The clones that generated correct-sized PCR products were selected for miniprep plasmid DNA isolation using a Qiagen kit. The nucleotide sequences of the coding regions were confirmed by sequencing using appropriate primers. Larger quantities of DNA were isolated using a maxiprep system to enable large-scale transfections to be carried out.

### Generation of the Chimeric Molecules

Small-scale transient transfections were performed by co-transfecting antibody heavy and light chain expression vector DNA (MoC601VH HuG4LIC and MoC601VK HuKLIC, 0.5 μg DNA each) into 1 ml ExpiCHO suspension cells in 24-well plates using ExpiFectamine CHO reagent. The ExpiCHO transfections were performed as duplicate well transfections and included wells for a HuG4K expression control. The transfected ExpiCHO supernatants were cultured for seven days, then harvested and clarified by centrifugation. The antibody concentrations in the supernatants were quantified on the Octet RED384 (ForteBio).

Expression levels for the MoC601 HuG4K antibody were 145 μg/ml and 155 μg/ml for the duplicate wells. Supernatant samples were also subjected to reducing SDS-PAGE where bands representing the heavy and light chains could be clearly seen relative to supernatant from mock-transfected cells. Binding of MoC601 HuG4K to human complement protein C6 was confirmed in an ELISA using expression supernatants.

Expi-293 cell transient transfections were carried out for MoC601 HuG1KFab (vectors MoC601VH HuG1FabLIC and MoC601VK HuKLIC) at 1 ml scale in a 24-well plate using Expifectamine 293 Reagent. After four days, the presence of Fab molecules in the supernatants were demonstrated by SDS-PAGE and their expression level estimated at 25 μg/ml from the gel. The expression supernatant was tested in an ELISA to confirm that MoC601 HuG1KFab was able to bind to C6.

A large-scale transfection in Expi-CHO cells was carried out to enable purification of the MoC601 HuG4K antibody. The MoC601 HuG1KFab large-scale transfection was performed in Expi-293 cells.

Quality control of the purified protein included aggregation analysis by SEC-MALS. The data generated showed no significant signs of aggregation and measured molecular weights are in good agreement with the expected molecular weights of the IgG4K monomer (145 kDa) and Fab (47 kDa) determined from their amino acid sequences.

The purified proteins were also analysed by mass spectrometry. The masses detected were sequence matched, allowing for standard modification and the expected Fc glycosylation.

Endotoxin levels were below the limits of detection for both MoC601 HuG4K (<0.0172 EU/mg) and MoC601 HuG1KFab (<0.0357 EU/mg), with the different values quoted reflecting the protein concentrations tested in the assay. MoC601 HuG1KFab was successfully crystalized and the structure of the Fab solved to 2.06 Å resolution by X-ray diffraction analysis.

### Biophysical Analysis of the Chimeric Antibody MoC601 HuG4K

Some biophysical analyses were performed for the chimeric antibody, MoC601 HuG4K. Firstly, the melting and aggregation temperatures (Tₘ, T_{agg}) were determined using the Uncle instrument. The sample temperature was increased whilst the unfolding of the proteins was monitored via changes in intrinsic fluorescence and the degree of aggregation by static light scattering. The first transition in the fluorescence curve gives a Tₘ value of 62.5 C. Further transitions in the fluorescence curve were not relevant to the Tₘ since static light scattering measurements indicated that aggregation had accompanied the first transition. The Tₘ of the chimeric antibody does not meet the LifeArc criterion of >65 C. A control antibody, Secukinumab, is included in each experiment and here had a Tₘ value of 70.5 C. Freeze-thaw analysis was also performed. A sample of the purified chimeric antibody was subjected to 10 cycles of 15 minutes at −80 C followed by thawing for 15 minutes at room temperature. Samples were analysed by SEC-MALS to check for aggregation. MoC601 HuG4K showed 3.5% aggregates after freeze-thaw stress, which passes the LifeArc strict criteria but not the ideal criteria.

In the heat stress test, samples were incubated at room temperature, 37 C and 50 C for one month then subjected to SEC-MALs analysis relative to a 4 C control. In this instance, the majority of the chimeric antibody in the 50 C had precipitated out of solution so this sample was not analysed by SEC-MALS. The room temperature and 37 C samples did meet the LifeArc ideal criteria as they did not contain detectable aggregates, but the antibody failed the heat stress test overall due to its precipitation at 50 C. MoC601 HuG4K was predicted to have a neutral isoelectric point (pI) of 7.24, which could be the cause of the precipitation. pI analysis of MoC601 HuG4K was performed using capillary isoelectric focusing (cIEF). This technique allows antibodies to be separated according to their pI using a pH gradient across the capillary. From these data, MoC601 HuG4K has lower pI than that predicted: the isoelectric points vary from 6.60-7.02 with the pI of the main isoform being at 6.84. Thus, most of protein comprises isoforms with pI values outside of the neutral pH range and a neutral pI is unlikely to be the sole cause of insolubility issues.

Cross-Interaction Chromatography using bulk purified human polyclonal IgG is a technique for monitoring non-specific protein-protein interactions and can be used to discriminate between soluble and insoluble antibodies. An elevated Retention Index (k′) indicates a self-interaction propensity and a low solubility. The k′ value for the MoC601 HuG4K was 0.0216, which is within the desirable range (<0.05).

### C6 Binding Activity of Chimeric Molecules

Prior to the generation of the chimeric molecules, the binding ELISA was optimised using purified mouse 1C9 antibody. The ELISA method published in Lin et al., 2020, uses human C6 protein at a concentration of 1 nM (0.12 μg/ml) to coat the ELISA plate wells. Since this is lower than the concentrations generally used, coating concentration of 0.5 μg/ml and 1.0 μg/ml were also tested. Binding of the mouse antibody (designated MoC601 MoG1K) was detected with an HRP-conjugated goat anti-mouse IgG (Fc-specific) reagent in an ELISA assay. The data generated showed that, in common with the published data, the 0.12 μg/ml C6 coating concentration gives a very shallow binding curve. This is likely to make the assay unsuitable for differentiating between the binding of different humanized variants to C6. In contrast, C6 coating concentrations of 0.5 μg/ml and 1.0 μg/ml yield results which show a clear dose-response to MoC601 MoG1K antibody, with sigmoidal-shaped curves and a consistent EC₅₀ value (0.12 nM). These results were confirmed in a second experiment. Due to lower signal variation, 1.0 μg/ml was used as the C6 coating concentration in subsequent experiments.

The chimeric molecules, MoC601 HuG4K and MoC601 HuG1KFab, were tested for C6 binding in the form of expression supernatants. In this experiment, the human molecules were detected with an HRP-conjugated goat anti-human kappa chain reagent, which gave relative low signals. However, the binding of both chimeric molecules to C6 was confirmed. The EC₅₀ value for MoC601 HuG4K (0.43 nM) was higher than that of the mouse antibody (0.07 nM), though this was lower than in previous experiments. A further experiment was performed to find a more suitable anti-human antibody detection reagent. MoC601 HuG4K from expression supernatant was detected with three different secondary antibodies. Both anti-human IgG Fc-specific and Fab-specific detection gave higher signals than the anti-kappa chain reagent but the Fc-specific detection yielded a very low EC₅₀ (0.03 nM). Another experiment, which tested different antigen coating concentrations and different dilutions of secondary reagent, did not help to explain why the alternative methods of detection generate dissimilar EC₅₀ values. It is common to find that different secondary antibodies yield different plateau levels but they do not generally affect the EC₅₀ values. The anti-human IgG (Fab-specific) secondary reagent will be used in subsequent assays since it gives a similar EC₅₀ (0.30 nM) to the mouse antibody (0.12 nM) and shows a good signal strength.

The binding of the purified chimeric molecules, MoC601 HuG4K and MoC601 HuG1KFab, was also confirmed by ELISA, where the anti-human IgG (Fab-specific) secondary reagent was used. The control mouse antibody bound as previously (EC₅₀ 0.11 nM). The EC₅₀ for purified MoC601 HuG4K (0.27 nM) was similar to that obtained for the MoC601 HuG4K supernatant sample above. The purified MoC601 HuG1KFab binding curve had a better-defined sigmoidal shape than seen for the supernatant sample. The EC₅₀ value for the Fab molecule (0.48 nM) was higher than for the antibody, which would be expected since the Fab is unable to bind bivalently to the antigen on the well. A C6-binding assay was also set up on the Octet RED384 instrument. Initial experiments demonstrated that MoC601 HuG4K antibody could be stably loaded on Protein G biosensors and bound C6 in a concentration-dependent manner. Unfortunately, capture of the mouse antibody MoC601 MoG1K by the Protein G biosensors was low and unstable and the antibody did not bind at all to Protein A biosensors. However, MoC601 MoG1K could be stably loaded onto anti-mouse Fc (AMC) biosensors. Concentrations of the mouse and chimeric antibodies were adjusted to give similar levels of loading on to the AMC and

Protein G biosensors respectively in subsequent experiments. There was no non-specific binding of C6 protein to unloaded sensors or sensors loaded with an isotype control antibody. A full kinetic experiment was run for purified MoC601 MoG1K and MoC601 HuG4K antibodies using seven concentrations of C6 (0.16-10 nM) and association and dissociation steps of 20 and 15 minutes respectively. The mouse and chimeric datasets were processed by subtracting the no antigen controls and the data were fitted to a 1:1 binding model. Binding curves giving R_{eq}/Rₘₐₓ ratios>90% or showing poor fitting were excluded from analysis. A second full kinetic experiment was performed with an adjusted concentration range (six points, 0.27-5 nM) and an extended dissociation step of 20 minutes. Comparison of the kinetic parameters generated by the two independent experiments show that the mouse and chimeric antibodies have very similar affinities as seen in Table 3:

TABLE 3

| Antibody | kₐ (M⁻¹s⁻¹) | k_{d} (s⁻¹) | K_{D} (nM) | Experiment |
|---|---|---|---|---|
| MoC601 MoG1K | 7.3 × 10⁵ | 3.1 × 10⁴ | 0.42 | 1 |
| MoC601 HuG4K | 6.9 × 10⁵ | 2.5 × 10⁴ | 0.36 | |
| MoC601 MoG1K | 6.9 × 10⁵ | 2.0 × 10⁴ | 0.29 | 2 |
| MoC601 HuG4K | 6.9 × 10⁵ | 2.4 × 10⁴ | 0.35 | |

### Example 3

### Sequence Determination and Characterization of Humanized Antibodies

This example describes determining the amino acid sequences for various humanized anti-C6 monoclonal antibody variants and characterizing such antibodies.

Human VH and VK cDNA Databases

The protein sequences of human and mouse immunoglobulins from the International Immunogenetics Database 2009 (Lefranc, 2015) and the Kabat Database Release 5 of Sequences of Proteins of Immunological Interest (last update 17 Nov. 1999) (Kabat et al., 1991) were used to compile a database of aligned human immunoglobulin sequences containing 10,406 VH and 2,894 VK sequences.

### Molecular Model of MoC601 Fv

Homology models of mouse C601 antibody variable regions were generated using the Antibody Prediction panel in Maestro 12.3 (Schrödinger). The mouse Fv structure 1E60, which was of high resolution and had both VH and VK sequences with high similarity to C601 VH and VK, was chosen to provide the structure of the framework regions upon which ten possible IMGT CDR loop models were generated. Throughout this work, the extents of the CDRs were according to the IMGT definition, as shown in FIGS. 2 and 3. The models were optimised using the One-step Protein Preparation wizard and Protein Reliability reports were generated to determine differences in model quality.

For all ten models of the mouse C601 Fv, residues that were within 4 Å of the CDR loops in any of the models were determined and designated as the “4 Å Proximity Residues”. By using all ten models, different possible orientations of the CDRs in solution are considered. In addition, residues within 4 Å of the VH-VK interface were determined. Comparison with the default Vernier, Canonical and Interface (VCI) residue set used in the analyses showed that the molecular modelling predicted extra interface residues. In the VH, there were four extra interface residues that had not already been identified as VCI or proximity residues. These were at IMGT positions 48, 49, 120 and 121. In the VK, extra interface residues were at positions 48, 49 and 51.

The crystal structure of the MoC601 HuG1KFab had not been solved by X-ray crystallography in time to assist with the humanisation design process. However, should there have been issues with the activity levels of the first-round variants, the crystal structure was available to aid the design process at that point. Comparison of the solved structure and the models showed good alignment of the framework regions. The CDR loops of the solved structure fell within the range of conformations suggested by the model. Many of the features of the models that were used to inform the variant design, for example, the presence of specific interactions between residues, were also observed upon retrospective examination of the solved structure.

### Human Framework Selection

Humanisation requires the identification of suitable human V regions. The sequence analysis program, Gibbs, was used to interrogate the human VH and VK databases with MoC601 VH and VK protein sequences using various selection criteria. Human VH sequences were ranked according to their identity to C601 VH at the Vernier, Canonical and Interface (VCI) residue and 4 Å Proximity Residue positions. The selection was further refined using overall sequence similarly and CDR length and humanized or patented sequences were removed from analysis.

For the top-ranking human frameworks, sequences of prospective C601 CDR-grafted versions (designated HA versions) were generated. Certain amino acids were highlighted within the sequences to aid identification of possible liability risks: unusual proline, cysteine or methionine residues and N-linked glycosylation, asparagine deamidation or aspartate isomerization risk motifs. Due to the risks present, accession numbers were marked for elimination or flagged. For the same human sequences, certain parameters were examined relating to their identity or similarity to C601 VH across the whole VH domain or just for the framework regions, VCI residues or 4 Å Proximity residues. Also examined were the predicted pI values of the prospective C601HA versions in order to ensure that none have neutral pI values and the percentage identity of the HA version to the nearest human germline V-region segment. We aim to generate humanized antibodies in which the percentage identity of the variable regions to the nearest human germline V-region segments is greater than 85% so identities score of <85% were marked in red in this column. However, successful humanisation is likely to require extra mouse residues to be incorporated into the human framework so an identity score of >90% would be desirable for the basic, CDR-grafted variable region, HA. Disappointingly, none of the entries gave identities>90%. Where the identity was greater than 85%, germline analysis (not shown) indicated that all the human candidates were derived from five different alleles of the same human germline gene, IGHV1-69, offering little choice in the selection process. Poor identity between the C601 and IGHV1-69 CDRs had caused the low human identity scores of the HA versions. It was decided to return to the output from the database analysis and rank the human VH sequence in a different manner.

Human VH sequences were ranked as previously but overall identity was given priority in the sorting process. The top 224 candidates were filtered to leave a set with unique HA sequences and the percentage identity of each of these HA variable regions to the nearest human germline V-region was determined. 26 human candidates yielded HA versions with a human identity of >90%. Eight human candidates were eliminated due to liability risks and two further candidates were flagged.

The sequence AB067157 was chosen as the human heavy chain donor candidate. This sequence scores highly in terms of identity of its HA version to human germline, has only one framework somatic mutation from its IGHV5-51*01 VH germline and has a similar CDR3 length to MoC601VH. This human VH had the minimum number of total mismatches at the VCI and 4 Å proximity residue positions: six mismatches at positions that are both 4 Å proximity and VCI residues, a further three mismatches at 4 Å proximity-only positions, four mismatches at VCI-only positions and one mismatch at one of the extra interface positions.

The selection of the human framework for the VK humanisation was more straightforward than for the VH since the standard ranking strategy provided a selection of suitable human candidates. Otherwise, the process followed was similar to that described for the VH above and allowed selection of a human kappa light chain donor candidate, X93759. This sequence scores highly in overall sequence identity to MoC601VK and the identity of its KA version to human germline. It has no framework somatic mutations from its IGKV3-11*01 VK germline and has the same CDR3 length to MoC601VK. For this human VK, there were seven mismatches at positions that are both 4 Å proximity and VCI residues, a further mismatch at a 4 Å proximity-only position, one mismatch at a VCI-only position and three mismatches at the extra interface positions.

### Design of C601 Humanized Heavy Chain Variants

Following identification of a suitable human framework, the synthetic protein and DNA sequences could be devised. The initial design of the humanized version of C601 VH was the grafting of CDRs 1, 2 and 3 from MoC601VH into the acceptor frameworks of AB067157, thereby creating variant C601HA. Considering all the key residue positions (VCI residues, proximity residues and extra interface residues), there are a total of 14 which are mismatched between MoC601VH and C601HA (positions 25, 39, 40, 53, 55, 66, 68, 69, 76, 78, 79, 89, 103 and 120). These residues were all back-mutated to the equivalent mouse residue in the humanized version C601HB.

Models were built for C601HAKA Fv so that potential interactions of the human residues in the unmatched proximity and VCI residues positions could be compared to those of the equivalent mouse residues in MoC601 Fv models. The aim was to predict of which of these residues might be most critical for binding and which are close in the structure so that they might need to be added as a group to have a positive impact on binding. This enabled the design of an ‘informed choice’ variant, C601HC, with fewer back mutations than HB. C601HC contains five back mutations.

In order to test the effects of the HC mutations individually, in each of the variants 5 HC1-HC5, one of the mutations was reverted to the human residue. The nine back mutations of HB that were not included in HC were added as single, additional back mutations to HC to create the variants C601HD to C601HL. Additionally, C601HM, contained a pair of these back mutations (N68 and G69). If HC did not bind as well as HB, variants HD-HM would allow identification of which residue differences were responsible. Unfortunately, inclusion of certain back-mutations, with the purpose of preserving antigen binding, does create liability risk motifs in some variants. These are the potential N-linked glycosylation site, NYS, at residue positions 66-68 and the asparagine deamidation risk motif, NG at residue positions 68 and 69. Note that the sequence NPS at positions 68-70 of variant HF does not constitute a potential glycosylation site since, in the motif NxS/T, x cannot be proline.

Finally, so-called framework swap variants were designed. In the event of any discrepancy in binding of HB relative to the chimeric heavy chain, these variants would be used to determine which part of the human framework was responsible for the reduction in activity. There are 17 residues differences between MoC601VH and C601HB. These residues were divided into four groups dependent on their position in the three-dimensional structure so that framework swap variants VH1-VH4 each contain three to six of the differing HB residues incorporated into the MoC601 VH sequence.

### Design of C601 Humanized Light Chain Variants

The framework from X93759 was used to design the humanized VK constructs. CDRs 1, 2 and 3 from MoC601VK are grafted into the acceptor frameworks of X93759 to generate the initial version of humanized C601, KA. There are 12 4 Å proximity/VCI residues which are mismatched between MoC601VK and C601KA (positions 1, 24, 39, 40, 48, 49, 51, 52, 53, 66, 67 and 87). These residues were all back-mutated to the equivalent mouse residue in the humanized version C601 KB.

As described for the VH above, model structures for MoC601 Fv and C601HAKA were examined and the ‘informed choice’ variant C601KC was designed to contain the seven back mutations thought most likely to affect binding. So that the effects of the KC mutations could be tested individually, in each of the variants KC1-KC7, one of the mutations was reverted to the human residue. The five back mutations of KB that were not included in KC were added as single, additional back mutations to KC to create the variants C601KD-KH.

As for the heavy chain, framework swap variants were designed in case there was a loss in binding activity for C601 KB compared to the chimeric light chain. There are 18 residues differences between MoC601VK and C601 KB. These residues were divided into four groups based on their position in the structure so that each of the framework swap variants had two to six KB residues substituted into the MoC601VK sequence.

### Generation and Properties of a Humanized Version of C601 Antibody

### Generation of Expression Vectors for the C601 Humanized Antibodies

The C601 humanized variants were to be expressed as full-length human IgG4K molecules. Bon Opus Biosciences designed DNA fragments that encoded each of the humanized and framework swap variants using their software algorithms to optimize the DNA sequences for expression in CHO cells. These variable region DNAs were synthesised, cloned into the appropriate pHuG4 LIC or pHuK LIC expression vectors and the vectors sequenced at Bon Opus Biosciences. The expression vectors were supplied to LifeArc as purified DNA preparations.

Bon Opus Biosciences provided each expression vector DNA in water at 1 μg/μl ready for transfection. This DNA was transformed into XL10-Gold ultracompetent cells to enable generation of bacterial glycerol stocks and the preparation of further quantities of plasmid DNA stocks using the QIAGEN Plasmid Miniprep and Maxiprep kits.

### Humanized Antibody Expression

Heavy and light chain vector combinations were co-transfected into ExpiCHO cells across four 96-well blocks with robotic assistance. The cells were then cultured for 7 days in serum-free medium, whereupon the conditioned medium containing secreted antibody was harvested. The concentrations of HuG4K molecules in the ExpiCHO cell supernatants were measured by Octet. Some of the antibodies were expressed poorly so supernatant samples containing less than 30 μg/ml antibody were re-quantified with greater accuracy by using lower range of antibody concentrations to make the standard curve. There was a large variation in expression levels from about 1 μg/ml (on the limit of detection) to about 200 μg/ml. It was noticeable that particular heavy or light chains had a tendency for high or low expression irrespective of which variant they are partnered with. Expression of control antibodies on each transfection plate indicated that there were no significant differences in expression from plate to plate. Each framework swap variant was expressed in combination with either MoC601VH or MoC601VK as appropriate.

The supernatant samples were normalised to 10 μg/ml, using the Hamilton robot. Where supernatants contained <10 μg/ml antibody, subsequent manipulations were carried out manually.

### C6 Binding Activity of the Humanized C601 Antibodies

The antibody expression supernatants, representing all the heavy and light chain combinations, were initially assessed in point-screen ELISA to enable the most ideal VH and VK variants to be identified for most detailed analysis. All the variants were tested at 2 μg/ml (13.3 nM) and at 0.1 μg/ml (0.67 nM) on two sets of duplicate plates. The two chosen concentrations fall on the plateau and on the slope of the binding curve for the chimeric antibody, with the 0.1 μg/ml assay likely to be most sensitive for antibodies that show a similar level of binding to the chimeric molecule. Titrations of the chimeric antibody and isotype control were included on each plate to verify the integrity of the assay. As previously, 2 μg/ml chimeric antibody was sufficient to saturate the binding signal and 0.1 μg/ml gave a signal towards the top of the slope of the sigmoidal curve. The most noticeable feature of the data is that all the of the variants involving the light chain KC2 showed very little binding even when tested at 2 μg/ml. This suggests that the back mutation to incorporate the mouse residue H40 is key to binding since H40 is present in KC but not KC2. Differences in binding performance between the heavy chains are more subtle and are best seen at the lower antibody concentration. Binding of HB-containing variants is generally low but tends to be worst for those antibodies that expressed poorly. This could be due to inaccurate antibody concentration or a low stability affecting both expression and binding. Most antibodies involving HC2 bound poorly and some of these also showed low expression so it is likely that the back mutation N40 is needed for optimum stability and binding. Antibodies using the HC3 heavy chain (missing back mutation Q55) also tended give a lower binding signal. As mentioned in the previous section, each framework swap variant has been expressed in combination with either MoC601VH or MoC601VK as appropriate. These supernatant samples were also included in the ELISA and all showed a good level of binding (A_{650 nm} for 0.1 μg samples of 1.4-1.6). These variants were designed to pinpoint the cause should there have been a loss of binding across all of the humanized variants.

From the point screening data, some of the best humanized VH variants (HC, HC1, HC5 and HF) were chosen. Full dose response ELISA data was obtained for supernatant samples of each of these chains in combination with all of the humanized light chains, except for KC2 which performed poorly in the point-screen ELISA. The samples were tested as single dilution series rather than in duplicate to enable a larger number of antibodies to be compared. The binding curves and EC₅₀ values for all antibodies tested were very similar and any slight differences could be accounted for by inaccuracies of quantification and the expected error limits of the ELISA. Also tested at this time were the best humanized VK variants (KA, KC, KC1, KC4, KE) in conjunction with all heavy chain variants except HB, HC2 and HC3, which did not perform well in the point-screen ELISA. All the antibodies gave very similar binding.

Since the ELISA failed to resolve any differences in C6 binding between the humanized variants, selected antibodies were tested in C6 binding on the Octet. Screening on the Octet was carried out at a single C6 concentration of 2.5 nM with 20 min association and dissociation steps. Antibodies were loaded onto 16 Protein G biosensors from diluted supernatant samples so that a set of humanized variants could be compared to the chimeric antibody and an irrelevant HuG4K control. Using a single concentration of antigen cannot provide accurate kinetic parameters but rather can show trends in data. One measure of association is found from the ratio of signals for the amount of C6 bound during the association phase over the amount of antibody loaded. The data can also be processed by subtracting the trace for HuG4K isotype control and fitting the curves to a 1:1 model to generate on- and off-rates. Fitting the dissociation curves in isolation to obtain the dissociation rate is expected to be more accurate.

Initial sets of humanized variants tested were the non-excluded heavy chains in combination with KC and the non-excluded light chains in combination with HC (based on ELISA data). Following these comparisons, HF and KC4 were judged to be amongst the best performing heavy and light chains so further experiments compared the light chains in combination with HF and the heavy chains in combination with KC4. As mentioned above, the parameters generated from analysis of binding of a single concentration of antigen are not accurate but visualising the data in this way allows trends to be noted.

Assessment of the heavy chain variants suggests that HF, HG, HH and HI perform better than HC so that addition of the extra back mutations in these variants may increase binding. Conversely, HC1 and HC5 variants appear to bind as well as HC despite each containing one fewer back mutation, suggesting that these murine residues are not necessary for binding. Amongst the VK variants, KC4 show superior binding to KC meaning that the human residue L52 is more favourable than the mouse R52. Other variants with one fewer back mutation than KC (KC1, KC3 and KC7) show similar binding to KC.

### Thermal Stability Testing of the Humanized C601 Antibodies

In order to see if it was possible to differentiate between the humanized variants in term of their stability, a thermal stability assay was carried out. In this assay, the IgG supernatants, representing a selection of variants, were diluted to an approximate EC₈₀ concentration of 0.33 nM, which is a concentration expected to fall towards the top of the linear portion of the ELISA antigen-binding curve. The samples were incubation at different temperatures for 10 min and then assayed for C6 binding by ELISA. The ELISA signals were normalized to the signal for the 35 C sample and plotted against temperature for each antibody. The LifeArc standard for the assay is that the antibodies should retain binding activity to at least 65 C. However, as shown in the upper panel, none of the antibodies retain>80% binding activity at this temperature and the chimeric antibody, MoC601 HuG4K, shows very low binding after incubation at 65 C. Some of the variants tested did show improved stability over the chimeric antibody. The temperature at which an antibody retains 50% binding activity can be determined as a method of ranking the variants. It is striking that the variants with higher stability than MoC601 HuG4K all used the KC4 light chain.

The mouse framework residue R52 was included the KC variant since modelling and, later, the MoC601 HuG1KFab crystal structure determination suggested that R52 has a salt bridge interaction with D116 in CDR3 of the VH. Such an interaction would have potential to influence binding and stability. It was therefore unexpected that the substitution of the human residue L52 into KC to create KC4 would result in a variable region that supports greater binding and stability. It seems likely that, in the absence of the salt bridge to D116, the conformation of the VH CDR3 allows a tighter interaction with the C6 antigen such that the R52L mutation could be considered a form of affinity maturation. With regard to stability, there is a potential Van de Waals interaction between the two aromatic rings of VH Y114 and VK Y55 in the interface. The side chain of R52 extends close to the tyrosine residues and appears to interfere with their interaction whereas the shorter side chain of L52 allows a stronger interaction between the aromatic rings.

### Choice of the Humanized C601 Antibodies for Mid-Scale Purification

From the variants already tested, the antibody C601HFKC4 HuG4K (aka 3713) has the best properties in terms of C6 binding in the Octet assays and thermal stability. However, 14 of the variants were selected for purification so that their functional activity in complement inhibition could be compared.

The mid-scale expression and purification process was designed for the purification of small amounts of antibody in a short time period. The variants were expressed in ExpiCHO for three days at 50 ml scale and purified via a one-step, affinity chromatography method.

In addition, some further VH and VK variants were designed. Some of the heavy chain variants contain a potential N-linked glycosylation site, NYS, at residue positions 66-68. Two VH domains lacking this motif are HC4 and HF. Data indicate that HF is preferable to HC4 in terms of expression, binding and thermal stability so additional VH variants were based on HF. Both HC1 and HC5 variants appear to bind as well as HC despite containing one fewer back mutation and this was seen more consistently for HC1. Thus, the HC1 mutation (A25G) was added into HF to increase its human germline identity and generate variant HN. Adding the extra back mutations of HG, HH and HI may increase binding over that of HC. It was decided, since they are close in the structure, to add mutations of HH (V76A) and HI (178L) together into HF to form HO. Finally, all three changes were made in one variant, HP.

It was shown above that the KC4 light chain was essential for improvement of thermal stability relative to the chimeric molecule and should, therefore be used as the basis for any further variants. Since the binding of KC4 was superior to KC and the chimeric antibody, adding extra mouse back mutations was not necessary. Data suggested that not all the back mutations in KC were needed for its level of binding so a further VK variant (KI) was designed with one fewer back mutation than KC4. This variant had E1 rather than Q1 as seen in KC1.

### Generation of Expression Vectors and Expression of the Additional Humanized C601 Variants

The vectors C601HN HuG4LIC, C601HO HuG4LIC and C601HP HuG4LICs were produced by site-directed mutagenesis of C601HF HuG4LIC using the primer(s) ‘A25G_for’ and/or ‘V76A_I78L_for. The vector C601KI HuKLIC was similarly produced using primer ‘Q1E_for’ and C601KC4 HuKLIC as the template. For correctly mutated clones, the nucleotide sequences of the coding regions were confirmed using sequencing primers and bacterial glycerol stocks were generated.

50 ml transfections were carried out with combinations of the new vectors, C601HF HuG4LIC and C601KC4 HuKLIC to express the new antibody variants. Supernatant samples were tested on the Octet to ensure that the expressed antibodies could bind C6 antigen (data not shown) before the antibody was purified from the supernatants by the one-step, affinity chromatography method.

Humanized C601 Antibodies from Mid-Scale Purifications

Of the 14 original variants and 7 new variants expressed at the 50 ml-scale, 20 antibodies were successfully purified. Expression of C601HNKI HuG4K appeared to be low and insignificant levels of antibody were seen in the fractions eluted during affinity chromatography.

All the antibody preparations were subjected to endotoxin testing. The levels of endotoxin were largely undetectable and, in these cases, the maximum EU/mg values given in the table are a function of the concentration at which the proteins were tested. The majority of each preparation was used for functional assessment in complement inhibition assays.

Some of the antibody variants have a potential N-linked glycosylation site in the framework 3 of the VH. In order to ascertain if glycosylation occurs at this site, two antibodies were analysed by mass spectrometry. These were C601HCKC4 HuG4K, with a potential glycosylation site in VH framework 3, and C601HFKC4 HuG4K (aka 3713), without glycosylation site. The heavy chains of both antibodies could be sequence-matched, allowing for the expected Fc glycosylation. Thus, there is no evidence to suggest that glycosylation occurs within the VH of C601HCKC4 HuG4K.

C5, C5b6 and C6 Binding Activity of the Humanized C601 Antibodies from Mid-Scale Purifications

The 20 purified antibodies were tested by ELISA for their abilities to bind human complement proteins C5, C5b6 and C6. The antibodies were divided into two sets for this analysis. MoC601 HuG4K and HuG4K isotype controls were included alongside each set, as was C601HFKC4 HuG4K (aka 3713) expression supernatant from the original transfection as an additional positive control. In addition, three humanized antibodies (C601HFKC4 HuG4K, C601HPKI HuG4K and C601HPKC4 HuG4K) were included within both sets to allow better comparison between the plates. The antibodies all show specificity for C6 with no detectable binding to C5. All humanized antibodies bind to C6 and to C6 as part of the C5b6 complex. The lower plateaus of the C5b6 complex binding curves are explained by less C6 protein being available for binding when wells are coated with C5b6 complex as opposed to C6 alone. For each antigen, the antibodies have similar binding curves with the ELISA failing to resolve any differences between them, as seen previously. The EC₅₀ values are included in the figure but slightly different plateau levels observed for the antibodies are influencing the apparent EC₅₀ values. However, it was noted that the EC₅₀ values tended to be lower on the plates for the second set of antibodies. For this reason, a subset of the 20 antibodies was tested in a further ELISA for binding to C6 and C5b6 complex.

The antibodies were compared in the binding of a single concentration of C6 on the Octet. Antibodies were captured on Protein G biosensors. Association of C6 at 2.5 nM was measured for 20 min, followed by a 20 min dissociation step. Some antibodies were tested in duplicate within each run or across the two runs, with allows the level of variation for identical samples to be judged. Data for each parameter are colour-coded across the two runs to aid comparison. Although this kinetic screen is limited in sensitivity, it does show that most of the antibodies have association and dissociation rates that are similar to or better than the chimeric antibody.

For two of the best performing antibodies, C601HFKC4 HuG4K (aka 3713) and C601HFKI HuG4K, a full kinetic experiment was run using seven concentrations of C6 (0.16-10 nM) and association and dissociation steps of 20 min. The datasets were processed by subtracting the no antigen controls and the data were fitted to a 1:1 binding model. Binding curves giving R_{eq}/Rₘₐₓ ratios>90% or showing poor fitting were excluded from analysis. Data suggests that these humanized antibodies have affinities as good as or better than the affinity of the chimeric antibody:

TABLE 4

| Antibody | kₐ (M⁻¹s⁻¹) | k_{d} (s⁻¹) | K_{D} (nM) |
|---|---|---|---|
| MoC601 HuG4K (experiment 1) | 6.9 × 10⁵ | 2.5 × 10⁻⁴ | 0.36 |
| MoC601 HuG4K (experiment 2) | 6.9 × 10⁵ | 2.4 × 10⁻⁴ | 0.35 |
| C601HFKC4 HuG4k | 11.9 × 10⁵ | 1.3 × 10⁻⁴ | 0.11 |
| C601HIKC4 HuG4k | 11.0 × 10⁵ | 1.2 × 10⁻⁴ | 0.11 |

Melting Temperature Determination for the Humanized C601 Antibodies from Mid-Scale Purifications

For melting temperature determination using the Uncle instrument, the standard protocol requires antibody samples at 1 mg/ml. Since the majority of the mid-scale preparations had antibody concentrations less than 1 mg/ml, the experiment was conducted at 0.5 mg/ml and limited to those antibodies at concentrations≥0.5 mg/ml. Since these antibodies were affinity-purified but not subjected to a size-exclusion step, the preparations were expected to contain aggregates and this was confirm by the dynamic light scattering measurement taken at the start of the run (data not shown). As previously, the sample temperature was increased whilst the unfolding of the proteins was monitored via changes in intrinsic fluorescence and the degree of aggregation by static light scattering. Antibodies with Tₘ values lower than and similar to the chimeric antibody comprised light chains with the murine residue R52. Variants using VK variants KC4 or KI, both with L52, had Tₘ values of up to 69.0 C.

### C6 Humanized Antibodies Purified at Large-Scale

Based on the binding and melting temperature analyses and, importantly, functional assessment in complement inhibition, five antibodies were chosen for large-scale production to enable full kinetic analysis of C6 binding, complete biophysical evaluation and further functional assays to be performed. These antibodies were sequenced and have the following designations C601HFKC4 HuG4K (aka 3713), C601HFKI HuG4K, C601HPKI HuG4K, C601HOKC4 HuG4K and C601HPKC4 HuG4K. The variable region amino acid sequences for these 5 antibodies are shown in FIG. 4. As previously, endotoxin levels were for the most part undetectable and the maximum EU/mg values given in the table are a function of the concentration at which the proteins were tested.

### C5, C5b6 and C6 Binding Activity of the Five Lead Humanized C601 Antibodies

The large-scale batches of the five lead antibodies were compared to the purified chimeric antibody and the previous, mid-scale preparations for binding to C5, C5b6 and C6 in ELISA. There was no binding to C5-coated wells as expected. In binding to C5b6 and C6, curves for all the anti-C6 are overlaid and any differences between curves are within the limits of the accuracy of the ELISA as seen previously. For the large-scale preparations of the lead antibodies, calculated EC₅₀ values for binding to C5b6 ranged from 0.09 nM to 0.12 nM and were comparable to an EC₅₀ of 0.09 nM for the chimeric antibody. In binding to C6, these humanized antibody preparations had EC₅₀ values of 0.17-0.23 nM compared to 0.20 nM for the chimeric antibody. Thus, the chimeric and all five lead antibodies bind equally to C6 and C5b6 when assessed by ELISA.

### Determination of Kinetics Parameters for the Binding of C6 to the Chimeric and Five Lead Humanized C601 Antibodies

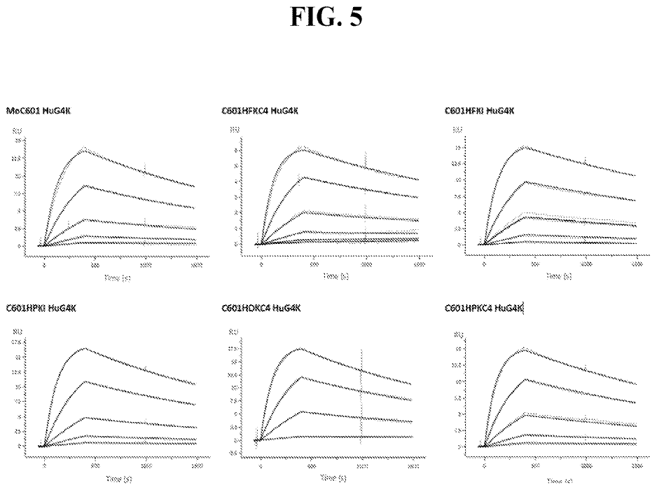

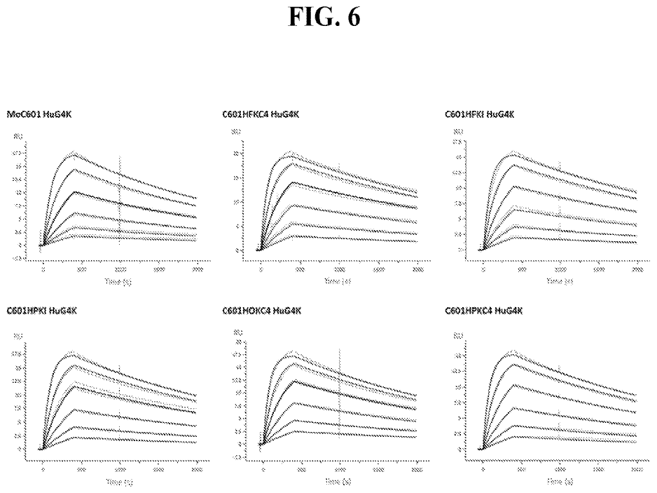

The kinetics of C6 binding was compared for the purified chimeric and lead humanized C601 using surface plasmon resonance (SPR, Biacore 8K). IgG were captured on a protein G sensor chip and binding of C6 measured using a three-fold concentration series of C6 from 0.03 nM-25 nM (FIG. 5). In a second experiment, a two-fold concentration series from 0.31 nM-20 nM C6 was used (FIG. 6). For all antibodies, binding of C6 occurred in a concentration-dependent manner and data could be fitted using a 1:1 model to calculate the association rate (kₒₙ), dissociation rates (k_{d}) and affinity constant (KD). The kinetic data are shown in Table 5, which indicates only small differences between the parameters determined from the two independent experiments.

TABLE 5

| | Experiment using 3-fold | | | | Experiment using 2-fold | | | |
|---|---|---|---|---|---|---|---|---|
| | concentration series of C6 (FIG. 5) | | | | concentration series of C6 (FIG. 6) | | | |
| Antibody | kₒₙ (M⁻¹s⁻¹) | k_{off} (s⁻¹) | KD (pM) | χ² (RU²) | kₒₙ (M⁻¹s⁻¹) | k_{off} (s⁻¹) | KD (pM) | χ² (RU²) |
| MoC601 HuG4K | 0.85 × 10⁶ | 4.48 × 10⁻⁴ | 527 | 3.07 × 10⁻² | 0.90 × 10⁶ | 4.13 × 10⁻⁴ | 459 | 6.48 × 10⁻² |
| C601HFKC4 HuG4K | 1.13 × 10⁶ | 3.89 × 10⁻⁴ | 343 | 0.73 × 10⁻² | 1.36 × 10⁶ | 3.06 × 10⁻⁴ | 226 | 12.4 × 10⁻² |
| C601HFKI HuG4K | 0.89 × 10⁶ | 3.08 × 10⁻⁴ | 348 | 6.43 × 10⁻² | 1.14 × 10⁶ | 3.17 × 10⁻⁴ | 279 | 7.03 × 10⁻² |
| C601HPKI HuG4K | 1.03 × 10⁶ | 4.45 × 10⁻⁴ | 433 | 0.98 × 10⁻² | 1.23 × 10⁶ | 3.61 × 10⁻⁴ | 295 | 11.5 × 10⁻² |
| C601HOKC4 HuG4K | 1.20 × 10⁶ | 4.92 × 10⁻⁴ | 411 | 2.83 × 10⁻² | 1.32 × 10⁶ | 3.75 × 10⁻⁴ | 284 | 8.26 × 10⁻² |
| C601HPKC4 HuG4K | 1.07 × 10⁶ | 3.97 × 10⁻⁴ | 373 | 3.39 × 10⁻² | 1.19 × 10⁶ | 3.55 × 10⁻⁴ | 299 | 3.71 × 10⁻² |

Across both experiments, the association rates for the humanized antibodies were similar to or higher than for the chimeric antibody whilst the dissociation rates tended to be lower for the humanized antibodies than the chimeric antibody. The affinities of the five humanized antibodies were slightly better than that of the chimeric antibody. For the humanized antibody demonstrating the highest affinity, C601HFKC4 HuG4K (aka 3713), the KD was 1.5- to 2-fold lower than the KD of the chimeric antibody. The range of KD values for C6 binding across the two experiments for the six antibodies is 0.23-0.53 nM. The reported KD value for the mouse 1C9 antibody is 2.1 nM (Lin et al., 2020), which is considered comparable considering the different SPR instrument and assay set-up.

### Aggregation Analysis of the Five Lead Humanized C601 Antibodies

Samples of the purified IgG were injected into a size-exclusion column in an HPLC system and analysed by multi-angle light scattering to determine the absolute molar masses and check for aggregation (see Table 6).

TABLE 6

| | | | | Mass |
|---|---|---|---|---|
| | | | Polydispersity | fraction |
| Antibody | Peak | Mw (kDa) | (Mw/Mn) | (%) |
| C601HFKC4 HuG4K | 1 | 138.2 ± 1.0 | 1.01 ± 0.01 | >99.5 |
| C601HFKI HuG4K | 1 | 139.7 ± 2.1 | 1.00 ± 0.02 | >99.5 |
| C601HPKI HuG4K | 1 | 140.9 ± 1.0 | 1.00 ± 0.01 | >99.5 |
| C601HOKC4 HuG4K | 1 | 141.2 ± 1.1 | 1.00 ± 0.01 | >99.5 |
| C601HPKC4 HuG4K | 1 | 139.5 ± 1.0 | 1.00 ± 0.01 | >99.5 |

The profiles show no signs of aggregation and an average molecular weight of about 140 kDa, which is the expected range for an IgG monomer in this analysis setup. The antibody is monodispersed (Mw/Mn<1.05). The mass recoveries are close to 100% (calculated mass over injected mass). This indicates good protein recovery and that the sample does not seem to stick to the column or contain insoluble aggregates, which would be retained by the guard column. Overall, the data suggest there are no aggregation concerns for the five lead humanized C601 candidate antibodies. Comparable data was previously obtained for the chimeric antibody.

### Analysis of the Five Lead Humanized C601 Antibodies by Mass Spectrometry

Mass spectrometry analysis was carried out on the purified humanized C601 antibodies, with the relative intensity of peaks being plotted against mass. The five purified humanized antibodies are of high purity with the intact analyses indicating a good match to the expected masses. The reduced analyses show that the mass of each chain is a good match to its expected sequence with standard modifications.

### Melting Temperature Determination for the Five Lead Humanized C601 Candidate Antibodies

Melting temperatures (Tₘ) were determined using the Uncle instrument. The first transitions in the fluorescence curves generate Tₘ values of 64.4-67.0 C for the five antibodies, with variants HFKC4 (aka 3713) and HFKI performing best (Table 7).

TABLE 7

| Antibody | Tₘ, C. | T_{agg}, C. |
|---|---|---|
| C601HFKC4 HuG4k | 67.0 | 69.5 |
| C601HFKI HuG4K | 67.0 | 70.0 |
| C601HPKI HuG4K | 65.0 | 67.3 |
| C601HOKC4 HuG4K | 65.0 | 67.0 |
| C601HPKC4 HuG4K | 64.4 | 66.6 |
| Secukinumab (control) | 69.7 | 73.5 |

Thus, these five humanized antibodies have higher melting temperatures than the chimeric antibody (62.5 C). Further transitions in the fluorescence curves were not relevant to the Tₘ since static light scattering measurements indicated that aggregation had accompanied the first transition. Dynamic light scattering measurement confirmed monomeric distribution of the starting samples and aggregation at end of the run (data not shown).

### Non-Specific Protein-Protein Interactions (CIC) for the Five Lead Humanized C601 Candidate Antibodies

Cross-Interaction Chromatography using bulk purified human polyclonal IgG is a technique for monitoring non-specific protein-protein interactions and can be used to discriminate between soluble and insoluble antibodies. An elevated Retention Index (k′) indicates a self-interaction propensity and a low solubility and a k′ value<0.05 is desirable. k′ values for the five lead antibodies ranged from 0.0129 to 0.0278 (Table 8), which are within the desirable range (<0.05).

TABLE 8

| | Retention Index |
|---|---|
| Antibody | (k¹) |
| Negative control (Rituximab) | 0.0296 |
| Positive control (CENT0607) | 0.4942 |
| C601HFKC4 HuG4K | 0.0129 |
| C601HFKI HuG4K | 0.0129 |
| C601HPKI HuG4K | 0.0276 |
| C601HOKC4 HuG4K | 0.0209 |
| C601HPKC4 HuG4K | 0.0199 |

### Solubility of Five Lead Humanized C601 Antibodies

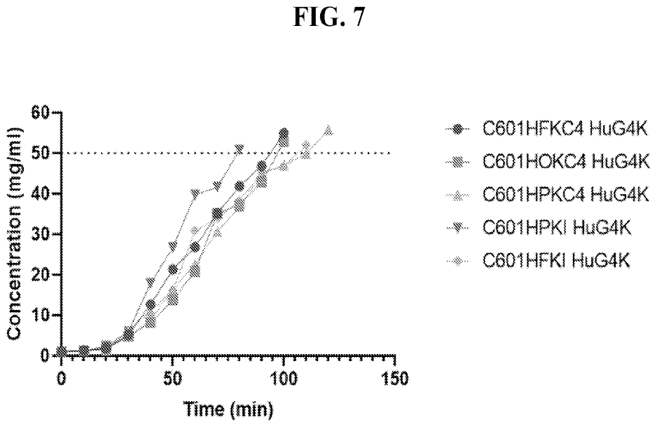

The lead humanized antibodies were subjected to sample concentration via solvent absorption concentrators (MWCO 7500 Da). Briefly, samples were placed into a concentration device and quantified periodically until the protein concentration was greater than 50 mg/ml or the dead volume was reached. All five antibodies could be concentrated to at least 50 mg/ml without apparent precipitation (FIG. 7). For four of the antibodies (HOKC4 excluded), the experiment was continued until the dead volume of the concentrating unit was reached. At this point, antibody concentrations were 68 mg/ml or greater.

### Isoelectric Point Analysis of Five Lead Humanized C601 Antibodies

Analysis of the five antibodies was performed using capillary isoelectric focusing (cIEF). This technique allows antibodies to be separated according to their isoelectric point (pI) using a pH gradient across the capillary. The range of main isoelectric points are as follows:

| | | | |
|---|---|---|---|
| C601HFKC4 HuG4K | 6.40-6.48 | C601HFKI HuG4K | 6.38-6.49 |
| C601HPKI HuG4K | 6.27-6.49 | C601HOKC4 HuG4K | 6.37-6.49 |
| C601HPKC4 HuG4K | 6.38-6.49 | | |

The isoelectric points of all five antibodies are below neutral pH, as tends to be the case for human IgG4 antibodies, and should not cause insolubility issues. The equivalent range for the chimeric antibody was at slightly higher pH (6.68-6.84; Section 4.2.3).

### Freeze-Thaw Stress Analysis of the Five Lead Humanized C601 Antibodies

Samples of the purified candidate antibodies were subjected to 10 cycles of 15 minutes at −80° C. followed by thawing for 15 minutes at room temperature. Samples were analysed by SEC-MALS to check for aggregation. The amount of aggregation caused by the freeze-thaw was 2.5-4.6% for the humanized C601 antibodies so all pass the strict QC criterion but are not behaving ideally. The best performing antibodies, C601HPKI HuG4K and C601HFKC4 HuG4K (aka clone 3713), showed less aggregation than the chimeric antibody (3.5%).

### Heat-Induced Stress Analysis of the Five Lead Humanized C601 Antibodies

Samples of purified antibodies were exposed to 4 C, room temperature, 37 C and 50 C for 28 days. Samples were then analysed by SEC-MALS to check for aggregation (Table 9).

TABLE 9

| | | | | | | | | Mass |
|---|---|---|---|---|---|---|---|---|
| | | | | Polydispersity | Mass | Aggregate | Degradation | eluted |
| Antibody | Sample | | Mw (kDa) | (Mw/Mn) | fraction (%) | (%) | (%) | (ug)* |
| C601HFKC4 | 4 | C. | 140.3 ± 0.9 | 1.00 ± 0.01 | 100 | — | — | |
| HuG4K | Room | | 138.8 ± 1.5 | 1.00 ± 0.02 | 100 | — | — | |
| | temperature | | | | | | | |
| | 37 | C. | 140.9 ± 0.8 | 1.00 ± 0.01 | 100 | — | — | |
| | 50 | C. | 141.4 ± 1.3 | 1.00 ± 0.01 | 90.7 | 3.8 | 5.4 | 10.5 |
| C601HFKI | 4 | C. | 140.3 ± 1.6 | 1.00 ± 0.02 | 100 | — | — | |
| HuG4K | Room | | 140.2 ± 1.6 | 1.00 ± 0.02 | 100 | — | — | |
| | temperature | | | | | | | |
| | 37 | C. | 139.5 ± 1.7 | 1.00 ± 0.02 | 100 | — | — | |
| | 50 | C. | 148.7 ± 1.8 | 1.00 ± 0.02 | 79.4 | 13.1 | 7.5 | 9.0 |
| C601HPKI | 4 | C. | 139.9 ± 1.2 | 1.00 ± 0.01 | 100 | — | — | |
| HuG4K | Room | | nd | nd | nd | nd | nd | |
| | temperature | | | | | | | |
| | 37 | C. | 140.1 ± 1.3 | 1.00 ± 0.01 | 100 | — | — | |
| | 50 | C. | 884.3 ± 25.4 | 1.40 ± 0.05 | 41.3 | 50.9 | 7.8 | 6.8 |
| C601HOKC4 | 4 | C. | 139.1 ± 0.9 | 1.00 ± 0.01 | 100 | — | — | |
| HuG4K | Room | | 137.8 ± 1.4 | 1.00 ± 0.01 | 100 | — | — | |
| | temperature | | | | | | | |
| | 37 | C. | 139.3 ± 0.8 | 1.00 ± 0.01 | 100 | — | — | |
| | 50 | C. * | nd | nd | nd | nd | nd | 3.3 |
| C601HPKC4 | 4 | C. | 140.5 ± 1.4 | 1.00 ± 0.01 | 100 | — | — | |
| HuG4K | Room | | 140.0 ± 1.2 | 1.00 ± 0.01 | 100 | — | — | |
| | temperature | | | | | | | |
| | 37 | C. | 141.4 ± 1.3 | 1.00 ± 0.01 | 100 | — | — | |
| | 50 | C. * | nd | nd | nd | nd | nd | 3.4 |

QC criteria: Ideally <0.5% aggregates (room temperature sample), <1% aggregates (37 C.) and <5% aggregates (50 C.) compared to unstressed sample after 1 month
Strictly <1% aggregates (room temperature sample), <5% aggregates (37 C.) and <10% aggregates (50 C.) compared to unstressed sample after 1 month

As was seen for the chimeric antibody, there were no aggregation concerns for the humanized C601 antibodies following incubation at room temperature or 37 C.

When the heat-induced stress test was carried out for the chimeric antibody, visible precipitate was apparent that the end of the incubation. In this experiment with the humanized antibodies, 50 C incubations were performed using a PCR instrument and a heating block. Data included in Table 10 relates to the heating block samples as these were available for all antibodies. Samples of the chimeric antibody were included and again showed a clear precipitate. No visible precipitate was seen with the humanized antibodies but other issues were found. Firstly, for three of the antibodies, the mass of antibody eluted from the column was significantly lower than expected. This is likely to be due to the presence of insoluble aggregates, which do not pass through the guard column. For C601HOKC4 HuG4K and C601HPKC4 HuG4K, only a third of the expected IgG was eluted so analyses of the monomer and aggregate peaks would not be valid. In the case of C601HPKI HuG4K, no antibody was eluted from the column for the sample incubated on the PCR instrument. Two-thirds of the expected amount of C601HPKI HuG4K in the heating block sample was eluted but half of this was in the form of aggregates and degradation products. For C601HFKC4 HuG4K (aka clone 3713) and C601HFKI HuG4K, there was little or no loss due to insoluble aggregates but aggregates and degradation were seen within the eluted protein. The monomer peak accounts for >90% C601HFKC4 HuG4K so this antibody passes the strict QC criteria. <80% C601HFKI HuG4K was in the monomer peak so this antibody does not meet the QC criteria.

The biophysical analyses have been deliberately carried out in PBS as this represents the worse possible scenario in terms of protecting the antibody in the stress testing. It is probable that the buffer optimisation could be used to negate any propensity for aggregation and/or degradation, especially in the case of C601HFKC4 HuG4K.

### Serum Stability Assessment of the Chimeric and Five Lead Humanized C601 Candidate Antibodies

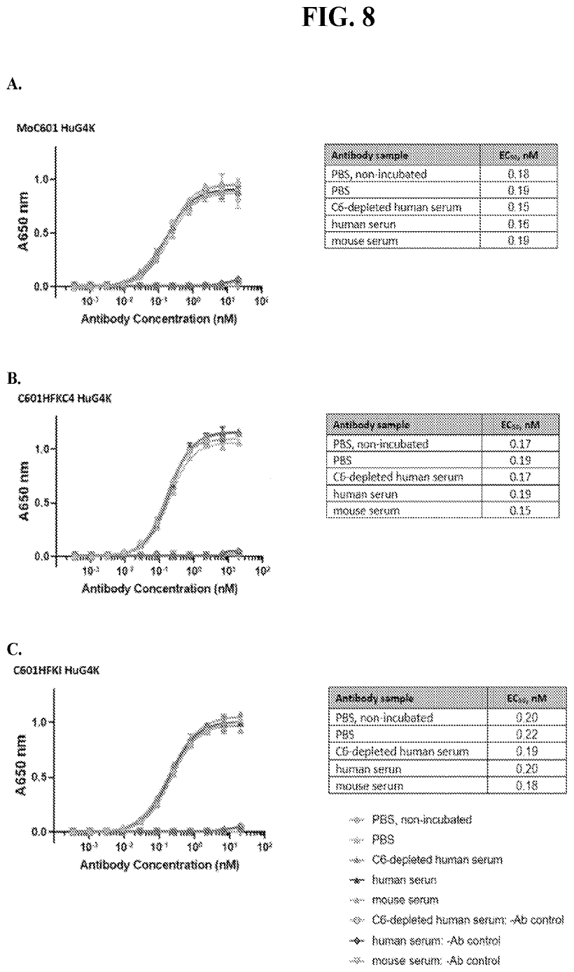

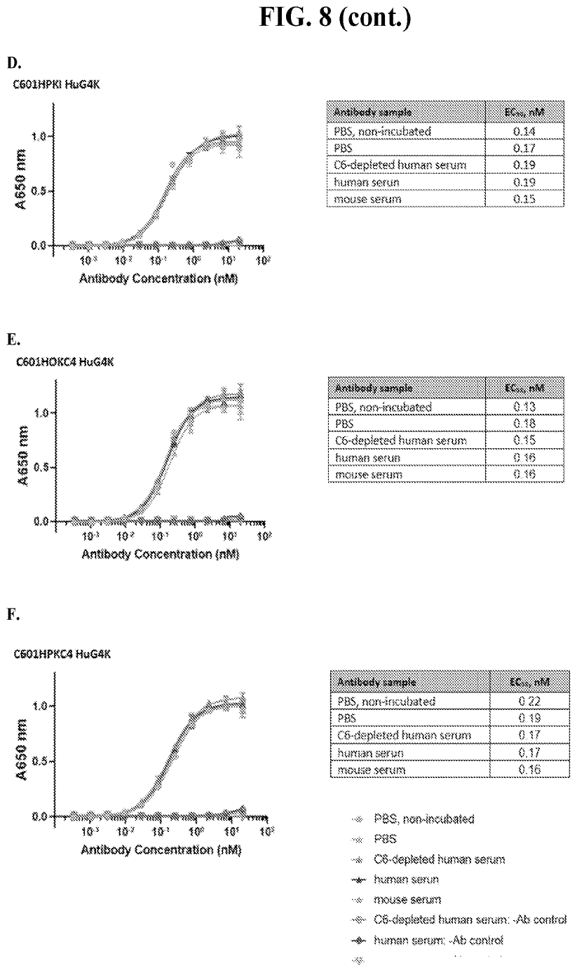

For the serum stability assessment, samples of the purified candidate antibodies are normally incubated in mouse, human and cynomolgus serum at 37 C for 28 days and then tested in an antigen-binding ELISA. Since the anti-C6 antibodies are expected to bind to both human and cynomolgus C6 but not mouse C6, we anticipated that incubating in human or cynomolgus sera would reduce ELISA binding due to occupation of the antibody binding sites by C6 protein from the sera. Therefore, the chimeric and humanized antibodies were tested with human and mouse sera and with human sera from which the C6 protein has been depleted. The C6-binding ELISA was then used to compare the binding abilities of the C601 antibodies that had been incubated in the 3 different sera to antibody samples that were incubated in PBS at 37 C or kept at 4 C (non-incubated). The ELISA results are shown in FIG. 8.

For each antibody, the binding of the serum-incubated samples to C6 is very similar to the binding of the PBS incubated and non-incubated antibody. Sera samples alone gave no signals in the ELISA. Therefore, the chimeric and five lead humanized C601 antibodies have retained their binding capabilities after being incubated in mouse serum and both C6-depleted and complete human serum. A reduction in ELISA binding had been expected for the antibodies in complete human serum, where the normal C6 concentration of 45-60 μg/ml is comparable to the test antibody concentration (100 μg/ml). It may be that the amount of C6 available for binding reduces during the 37 C incubation due to any instability of the protein. Additionally, the test antibodies are expected to bind bivalently to C6 antigen coated in the ELISA wells. Since a bivalent interaction will be tighter than that with free C6 in solution, the C6 from the sera may be displaced from the antibody binding site during the incubation step of the ELISA.

### SUMMARY

The binding affinity and biophysical properties of the five lead humanized antibodies are summarised and compared to those of the chimeric antibody in Table 10.

TABLE 10

| | Chimeric | | | | | |
|---|---|---|---|---|---|---|
| | antibody | Humanized antibodies | | | | |
| | MoC601 | C601HFKC4 | C601HFKI | C601HPKI | C601HOKC4 | C601HPKC4 |
| | HuG4K | HuG4K | HuG4K | HuG4K | HuG4K | HuG4K |
| Purified antibody | 218 | 290 | 60 | 46 | 209 | 98 |
| yield (mg/l) | | | | | | |
| C6 binding by | 0.20 | 0.19 | 0.22 | 0.17 | 0.21 | 0.23 |
| ELISA (EC₅₀, nM) † | | | | | | |
| C5b6 binding by | 0.09 | 0.11 | 0.10 | 0.09 | 0.11 | 0.12 |
| ELISA (EC₅₀, nM) † | | | | | | |
| Selectivity | Selective | Selective | Selective | Selective | Selective | Selective |
| (no C5 binding | | | | | | |
| by ELISA) | | | | | | |
| KD (pM) by SPR * | 527 | 343 | 348 | 433 | 411 | 373 |
| pI range | 6.60-7.02 | 6.26-6.60 | 6.19-6.61 | 6.19-6.69 | 6.16-6.60 | 6.17-6.60 |
| Tm (C.) | 62.5 | 67.0 | 67.0 | 65.0 | 65.0 | 64.4 |
| Solubility (mg/ml) | nd | >50 | >50 | >50 | >50 | >50 |
| 1 month 50 C. | Precipitation, | 3.8% | 13% | 51% | Insoluble | Insoluble |
| heat stress | not analyzed | aggregate, | aggregate, | aggregate, | aggregates | aggregates |
| (aggregation | | 5.4% | 7.5% | 7.8% | | |
| by SEC-MALS) | | degradation | degradation | degradation | | |
| Freeze-thaw stress | 3.5% | 2.8% | 4.5% | 2.5% | 3.8% | 4.6% |
| (aggregation by | | | | | | |
| SEC-MALS) | | | | | | |
| Non-specific | 0.0216 | 0.0129 | 0.0129 | 0.0276 | 0.0209 | 0.0199 |
| interactions | | | | | | |
| (k′) | | | | | | |
| Serum stability | Retained | Retained | Retained | Retained | Retained | Retained |
| | binding | binding | binding | binding | binding | binding |
| VH: % ID with | 66.3% | 89.8% | 89.8% | 88.8% | 87.8% | 88.8% |
| closest human | (IGHV1- | (IGHV5- | (IGHV5- | (IGHV5- | (IGHV5- | (IGHV5- |
| germline V- | 69*08) | 51*01) | 51*01) | 51*01) | 51*01) | 51*01) |
| segment | | | | | | |
| VH: LakePharma | 64.1 | 82.7 | 82.7 | 81.7 | 81.1 | 81.7 |
| T20 score | | | | | | |
| VL: % ID with | 62.5% | 84.1% | 85% | 85% | 84.1% | 84.1% |
| closest human | (IGKV3D- | (IGKV3D- | (IGKV3D- | (IGKV3D- | (IGKV3D- | (IGKV3D- |
| germline V- | 11*02) | 11*02) | 11*02) | 11*02) | 11*02) | 11*02) |
| segment | | | | | | |
| VK: LakePharma | 62.5 | 84.1 | 85.0 | 85.0 | 84.1 | 84.1 |
| T20 score | | | | | | |

For each variable region used in these antibodies, the table also contains the percentage identity to the closest human germline V-segment, as determined at www followed by “imgt.org/3Dstructure-DB/cgi/DomainGapAlign.cgi.” In addition, the LakePharma T20 score is given. The T20 score is calculated on a scale of 0-100, with a higher score indicating that a sequence has more characteristics of human variable region. Complete variable regions with a T20 score above 80, like those of the humanized candidates, are considered human-like.

The abilities of the five lead humanized and chimeric C601 antibodies to bind human C6 are comparable when tested in ELISA whereas Biacore experiments suggest that the humanized version may have slightly higher affinity. The biophysical properties of the five lead humanized C601 candidate antibodies are similar to or better than those of the chimeric C601 antibody. For the humanized candidates, assays measuring thermal stability of binding, melting temperature, solubility, isoelectric point, propensity for non-specific protein-protein interactions and serum stability did not indicate any issues. Aggregation was seen in response to freeze-thaw at a permitted but not ideal level. Although all five candidates performed better against heat stress than the chimeric antibody, various levels of aggregation and degradation were seen after incubation at 50 C for four weeks. The C601HFKC4 HuG4K (aka clone 3713) passed the strict criteria for this assay. Since the stress assays were conducted in PBS, buffer optimisation would be expected to address the tendency for aggregation. C601HFKC4 HuG4K was the best, or one of the best, humanized antibodies in each of the assays and has a good purification yield, adding to its drug-like characteristics.

### Example 4

### Humanized 3713 (C601 HFKC4) mAb

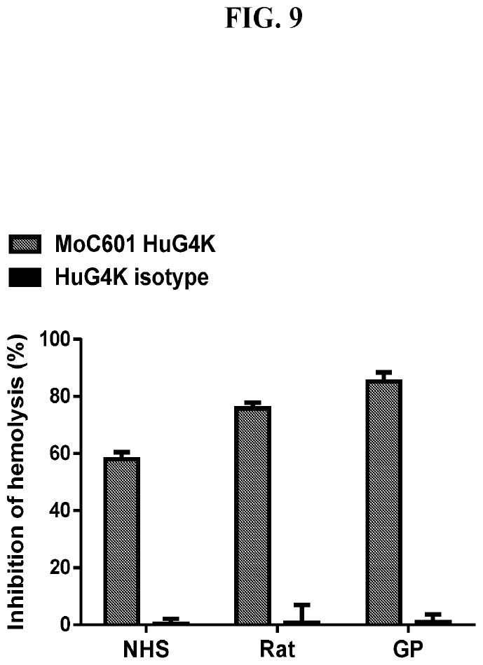

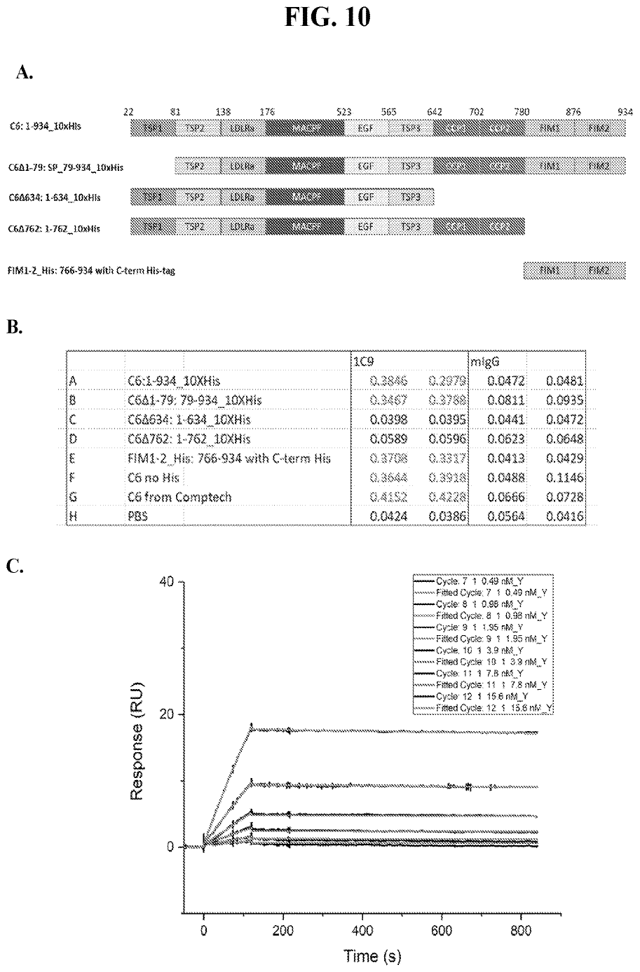

C6 is the complement component downstream to C5 for MAC assembly in the complement activation cascade. Targeting C6 selectively inhibits MAC without affecting C5a production, thus representing an attractive therapeutic approach. We previously reported the development of mAb 1C9, a mouse anti-human C6 mAb, and showed its efficacy in inhibiting MAC-mediated hemolysis both in vitro and in vivo. In this example, we further examined the cross-reactivity of mAb 1C9 to other rodents C6 (FIG. 9), mapped the binding domains on C6 for mAb 1C9 (FIG. 10), and completed its humanization process.

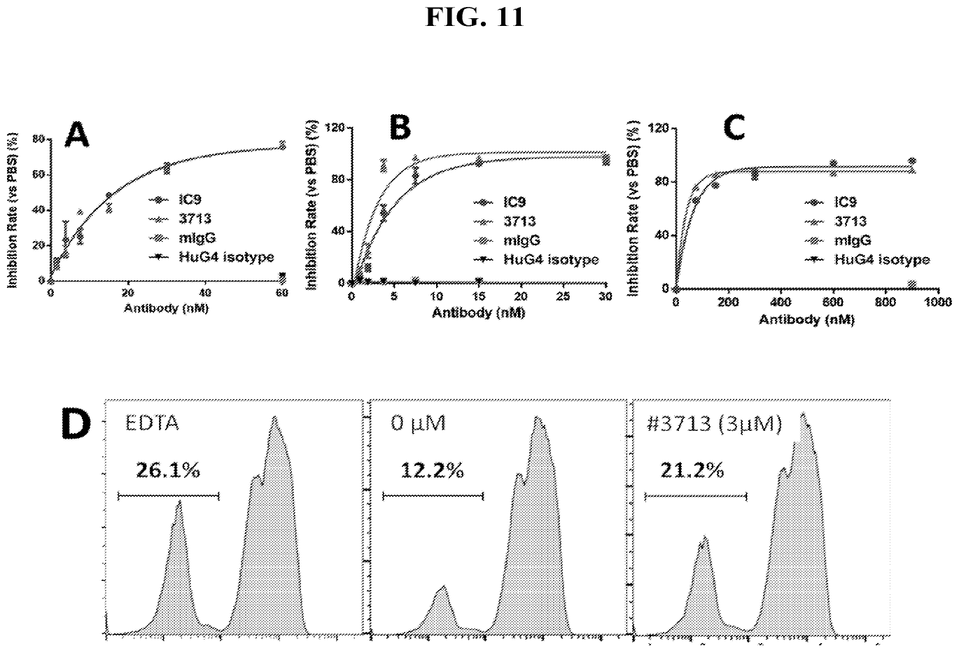

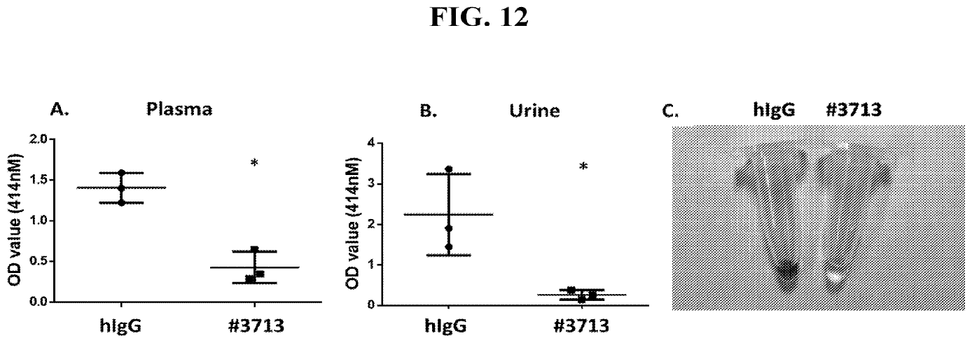

Anti-C6 mAb 1C9 was humanized using molecular modeling and complementarity-determining region grafting. After screening a library of 256 humanized variants (human IgG4s) with different combinations of humanized light and heavy chains in biophysical assays, clone 3713 (aka C601 HFKC4) showed the best developability profile, and an increased affinity against C6 when compared with the parental 1C9 mAb. This humanized 3713 mAb inhibited both human, monkey, and rat complement-mediated hemolysis in vitro (FIG. 11A-C), protected PNH patient RBCs from complement-mediated damage in vitro (FIG. 11D), and more importantly, it significantly reduced complement-mediated hemolysis in vivo in rats (FIG. 12). These results demonstrated the successful humanization of the anti-C6 mAb.

### Methods and Reagents

### Complement Hemolytic Assays

Sheep red blood cells (RBCs; Hemostat Laboratories, Dixon, CA, USA) were initially incubated at 37° C. for 30 min with a 1:800 dilution of rabbit anti-sheep RBC serum (MP Biomedicals, Santa Ana, CA, USA) in the GVB-E buffer (GVB; 10 mM barbital, 145 mM NaCl, 0.1% gelatin and 10 mM EDTA, pH 7.2) for sensitization. Approximately 5×10⁶ sensitized sheep RBCs (E^{sh} A) were incubated with 1% sera of human, non-human primate (NHP), rat or guinea pig in the presence or absence of different concentrations of various purified anti-C6 IgGs in gelatin veronal buffer containing Mg++ and Ca++ (GVB++; 10 mM barbital, 145 mM NaCl, 0.5 mM MgCl₂, 0.15 mM CaCl₂), 0.1% gelatin, pH 7.2±0.15; Boston BioProducts, Ashland, MA, USA) at 37° C. For negative controls, 5 mM EDTA was added to the tubes to inhibit complement activity.

After a 5-min incubation, the E^{sh} A were centrifuged and the supernatants were collected. The optical density was measured at 414 nm (OD414), and the following equation was used to calculate the percentage of hemolysis: Hemolysis (%)=[(A−B)/(C−B)]×100%. Here, A, B and C represent the OD readings of the sample with serum, the sample with serum and EDTA and the maximum hemolysis induced by water, respectively.

### 1C6 Binding Site Mapping Using Truncated C6 Proteins

ELISA plate wells were coated with 50 μl of different truncated C6 proteins in PBS (2 nM) and incubated overnight at 4° C. The plate was then washed and blocked with 1 mg/ml bovine serum albumin in PBS for 1 hr at room temperature. Next, different dilutions of the anti-C6 mAb (1 mg/ml) were added to each well and incubated at room temperature (RT) for another 2 hr. After washing, a horseradish peroxidase-conjugated goat anti-mouse IgG antibody was added, and the plate was incubated for 1 hour before development.

### Determination of the Interaction of 1C9 for C6 FIM Domains by SPR:

SPR studies were performed using a BIAcore T200 device (GE Healthcare, Marlborough, MA, USA) at 25° C. The binding assays were conducted in PBSP+ buffer, which contained 20 mM of phosphate (pH 7.4), 137 mM of NaCl and 2.7 mM of KC1, plus 0.05% surfactant P20. Next, mAb clone 1C9 at a concentration of 50 μg/mL in 10 mM sodium acetate (pH 4.5) was injected at a rate of 10 μL/min and amine-coupled to S-series CM5 sensor chips (BIAcore) using a standard amine coupling kit (BIAcore). The final density of 1C9 reached 350 RU. Two-fold series of purified FIM1-2 domains of C6 ranging from 0.975 nM to 500 nM were then injected at a rate of 30 mL/min over surfaces on which 1C9 had been immobilized. Each data point was repeated twice, and double referencing was applied to all detected traces using BIAevaluation software (version 6). Briefly, the data were referenced by subtracting an unmodified surface to correct the binding responses for the bulk refractive index change. Next, the response from an average of blank injections was subtracted from the data values to remove any systematic artifacts.

### mAb 1C9 Isotyping and Sequencing

The isotype of the 1C9 antibody was determined from hybridoma cell supernatant using a IsoStrip™ Mouse Monoclonal Antibody Isotyping Kit (Roche). RNA was isolated from a hybridoma cell pellet (RNeasy Kit, Qiagen) and reverse-transcribed (First strand cDNA synthesis kit, Cytiva). VH and VK cDNAs were each amplified by PCR using an array of leader sequence primers and a constant region primer using Phusion Flash High-Fidelity PCR Master Mix (Fisher Scientific). Correctly sized products were directly sequenced (Azenta Life Sciences) in both directions.

### Humanization and Biophysical Assays

Human VH and VK framework regions for grafting of 1C9 complementarity-determining regions (CDRs) were selected through sequence analysis and molecular modeling (Maestro, Schrödinger). Modeling was further used to inform the design of variants with framework substitutions. Variable region DNAs were synthesized and cloned into human IgG4 or kappa expression vectors (Bon Opus Biosciences, NJ). Vectors were transfected in all VH/VK combinations at 0.6 ml scale into ExpiCHO-S cells (Invitrogen), according to the manufacturer's protocol. IgG in expression supernatants was quantified (Octet RED96, Sartorius). Variants of interest were purified from larger-scale ExpiCHO-S expressions using PrismA, followed by size-exclusion chromatography, on AKTA Expressors (Cytiva Life Sciences). Purified protein QC included size-exclusion chromatography and multi-angle light scattering analysis (SEC-MALS) to show that antibodies were monodispersed with no detectable aggregates. Mass spectrometry confirmed purity and that intact proteins and component heavy and light chains were of the sizes predicted from their sequences. Endotoxin levels (nexgen-MCS, Charles River) were below the limit of detection (<0.1 EU/mg).

Binding ELISAs used Nunc Maxisorp 384-well plates, wells coated with C6, C5b,6 complex or C5 (Complement Technology Inc, TX) and goat anti-human IgG (Fab specific) peroxidase secondary antibodies (Sigma-Aldrich, A0293) for detection. For the thermal stability assay, IgG supernatants were diluted to their EC₈₀ concentration, incubated at different temperatures for 10 min and C6 binding measured by ELISA with the binding signal plotted against temperature. Kinetics of C6 binding were measured by capturing antibodies from supernatant samples using Protein G biosensors and performing a 20 min association step with 2.5 nM C6 and a 20 min dissociation step using the Octet RED96. Precise kinetic measurements were obtained by surface plasmon resonance (Biacore 8K, Cytiva). The IgG were captured on a protein G sensor chip. Binding of C6 measured using a three-fold concentration series of C6 from 0.03 nM-25 nM and data were fitted using a 1:1 model in two independent experiments.

The Uncle instrument (Unchained Labs) was used to monitor unfolding via intrinsic fluorescence and aggregation by static light scattering across a temperature gradient to obtain melting temperatures (Tₘ). Accelerated stress testing, with analysis by SEC-MALS, included incubation of antibody samples of at 4 C, room temperature, 37 C and 50 C for 28 days and, for freeze-thaw, subjecting samples to 10 cycles of 15 minutes at −80° C. followed by thawing for 15 minutes at room temperature. For the serum stability assessment, samples of antibodies were incubated in mouse, human and C6-depleted human serum at 37 C for 28 days then assayed for retention of C6 binding by ELISA in comparison to PBS samples incubated at 37 C or 4 C. Propensity for non-specific protein-protein interactions was assessed through cross-interaction chromatography, from which the Retention Index compares retention time on columns of uncoupled resin and resin coupled to bulk purified human polyclonal IgG; values close to zero (k′<0.05) suggest ideal behavior. Isoelectric points were determined by capillary isoelectric focusing using a PA800+ (Beckman Coulter). Solubility was judged by concentrating samples to at least 50 mg/ml without apparent precipitation using Vivapore solvent absorption concentrators (7500 kDa MWCO, Sartorius).

### PNH Patient RBC Protection Assay

A whole blood sample was obtained from a diagnosed PNH patient (deidentified) with consent following a Cleveland Clinic Institutional Review Board (IRB)-approved protocol. The RBCs were washed and resuspended in GVB-Mg-EGTA buffer at a concentration of 2×108/ml. Then RBCs were incubated with 50% acidified NHS (pH 6.4) in GVB-Mg-EGTA buffer at 37° C. for 15 min in the absence or presence of 3 μM of the humanized mAb 3713, or 10 mM EDTA (control). After incubation, RBCs were washed again, stained with an APC-conjugated anti-human CD59 mAb (Biolgend, CA) and followed by flow cytometric analyses.

### In Vivo Treatment Studies in Rats

Following a published protocol, complement-induced intravascular hemolysis was induced by intravenously injecting human RBCs into WT rats. The rats were then randomly divided into two groups with one group treated with 8 mg/kg of clone 3713 and the other with the same volume of vehicle (sterile PBS). Ten minutes after injection, rats were euthanized, blood samples were collected via intracardiac punctures, and urine samples were collected via intra-bladder punctures. After centrifugations, the plasma and urine samples were collected, and the extent of hemolysis in each was measured by quantitating the OD414 as a spectrophotometric measure of hemoglobin release.

### Results

mAb 1C9 Cross-Reacts with Rat and Guinea Pig C6:

We previously reported that 1C9 cross-reacts with monkey but not mouse C6, suggesting that it can be evaluated in non-human primates (NHPs) in vivo before a clinical trial in humans. But NHPs are becoming more and more expensive and difficult to obtain, we thus examined the potential cross-reactivity of 1C9 to rat or guinea pig C6 by determining if 1C9 inhibits rat or guinea pig complement-mediated hemolysis. We found that 1C9 potently inhibited hemolysis mediated by rat or guinea pig complement in a concentration-dependent manner, suggesting that this mAb cross-reacts with C6 in rats and guinea pigs in addition to NHPs.

### 1C9 Binds to the FIM Domains of C6

We then determined the domains of C6 that 1C9 binds to by ELISA using various recombinant C6 truncated proteins. These assays showed that 1C9 bound to all C6 protein variants containing the FIM domains except those that do not, suggesting that this mAb binds to the FIM domains of C6. We further validated the direct interactions between 1C6 and the FIM domains of C6 by SPR and found that 1C6 bound to the FIM domains with an affinity of XnM, which is comparable to the affinity measured by using the whole C6 protein.

### Design and Production of mAb 1C9 Humanized Variants as Human IgG4 Proteins

We determined the isotype of mAb 1C9 as mouse IgG1κ. We isolated total RNAs from the 1C9 hybridoma cells and specifically amplified the variable regions of the VH and VK after a reverse transcription. Molecular modeling and sequence analysis allowed the selection of human variable region frameworks to accept the 1C9 CDRs and multiple versions of the humanized 1C9 VH and VK domains were designed which maintained murine residues at different framework positions. We thus expressed a total of 276 distinct heavy and light chain combinations in a human IgG4,κ format by transient transfection of ExpiCHO-S cells. We also generated a chimeric version of 1C9 comprising the murine variable regions and human IgG4 and k constant regions as a control.

We then examined the C6 binding of normalized antibody expression supernatants by ELISA. For more than 100 variants, full-dose response curves did not show any differences in C6 binding compared to the chimeric antibody whereas kinetic analysis of C6 binding revealed some differences in association or dissociation rates. We found that some humanized variants showed improved stability over the chimeric antibody in a thermal stability assay and were prioritized when selecting 20 humanized antibodies for 50 ml-scale expression and purification by affinity chromatography.

The affinity-purified antibodies bound to free C6 and to C6 as part of the C5b6 complex but with no detectable binding to C5 by ELISA. All these candidates significantly inhibited human and rat complement-mediated hemolysis by an amount that was comparable to their parental 1C9 mAb at the concentration tested. Neither the mouse IgG or human IgG controls showed any inhibition, as expected. Based on these results, and antibody melting temperatures (Tₘ) as an indicator of stability, we chose the top five candidates for purification using affinity and size-exclusion chromatography and full characterization.

### Characterization of Five Lead Humanized Candidates

We confirmed the binding specificity of the five leads again by ELISA and compared the kinetics of C6 binding for the purified chimeric and lead humanized antibodies using SPR. The affinities of the five humanized antibodies (0.23-0.43 nM) were slightly better than that of the chimeric antibody (0.53 nM). We then carried out a panel of biophysical experiments to assess the developability of the candidates. The assessment was carried out in PBS rather than an optimized buffer in order to draw out any differences between the candidates' biophysical properties and identify issues that should be addressed through formulation. The results for the antibody that was ultimately selected as lead (clone 3713, aka C601 HFKC4) are shown in Table 11, in comparison to the data for the chimeric antibody, and it is apparent that clone 3173 exhibits greater stability.

TABLE 11
Properties of the chimeric and the lead humanized 1C9. A library of 276 humanized
variants were produced and analyzed, #3713 is identified as the lead candidate.

| | Chimeric | Humanised |
|---|---|---|
| | antibody | antibody 3713 |
| Binding properties | | |
| C6 binding by ELISA (EC₅₀, nM) | 0.20 | 0.19 |
| C5b6 binding by ELISA (EC₅₀, nM) | 0.09 | 0.11 |
| Selectivity (no C5 binding by ELISA) | Selective | Selective |
| KD (pM) by SPR | 527 | 343 |
| Production levels and biophysical properties | | |
| Purified antibody yield (mg/l) | 218 | 290 |
| pI range | 6.60-7.02 | 6.26-6.60 |
| Tm (C.) | 62.5 | 67.0 |
| Solubility (mg/ml) | nd | >50 |
| Aggregation in purified sample | <0.5% | <0.5% |
| 1 month 50 C. heat stress (aggregation by SEC-MALS) | Precipitation, | 3.8% aggregates, |
| | not analysed | 5.4% degradation |
| Freeze-thaw stress (aggregation by SEC-MALS) | 3.5% | 2.8% |
| Non-specific interactions (Retention Factor) | 0.0216 | 0.0129 |
| Serum stability | Retained | Retained |
| | binding | binding |
| Sequence identity with human V-segments | | |
| VH: % ID with closest human germline V-segment | 66.3% | 89.8% |
| VL: % ID with closest human germine V-segment | 65.6% | 88.9% |

Tₘ values were 64.4-67.0° C. for the five humanized antibodies, compared to 62.5° C. for the chimeric antibody and aggregation started to occur between 66.6 and 70.0° C., compared to 62.8° C. for the chimeric antibody. Following 28-day incubation at 50° C., the chimeric antibody sample contained visible precipitate but >90% of clone 3713 remained in the monomer peak. Clone 3713 also showed less aggregation in response to freeze-thaw than the chimeric antibody. The chimeric and humanized antibodies demonstrated ideal behaviors in cross-interaction chromatography, isoelectric focusing, serum stability, and solubility assessments.

### Clone 3713 Potently Inhibits Hemolysis Mediated by Activated Human, NHP and Rat Complement In Vitro

To validate the MAC-inhibiting activity of clone 3713, we evaluated its potency in inhibiting hemolysis mediated by activated complement using sera from human, NHP, or rat as the source of complement. We found that as expected, clone 3713 inhibited all the human, NHP and rat MAC-mediated hemolysis in a concentration-dependent manner.

Clone 3713 Protects PNH Patient RBCs from being Damaged by Activated Complement

One of the hallmarks of PNH is the MAC-mediated damage of the RBCs that lack CD55 and CD59 as a result of the GPI-anchor pathway deficiency in certain hematopoietic stem cells. We induced complement-mediated hemolysis using a PNH patient sample containing ˜26% of CD59-PNH RBCs in the absence or presence of 3 μM of clone 3713, then measured the survival of the CD59-PNH RBCs by flow cytometry. These assays showed that after complement activation, in the absence of clone 3713, CD59-RBC population was reduced to 12% from 26%, while in the presence of clone 3713, there were still 21% of CD59-RBCs existed in the patient sample.

### Clone 3713 Protects Rats in a Model of Complement-Mediated Hemolysis In Vivo

Finally, we used the same rat complement-mediated hemolysis model in our previously published report to evaluate the treatment efficacy of clone 3713 in vivo. In brief, after infusing human RBCs into healthy WT rats, we randomly divided them into two 2 groups and treated them with either clone 3713 or the same volume of PBS. We then measured the levels of free hemoglobin in both the plasma and urine after 30 min. These studies found that while the mock-treated rats showed massive intravascular hemolysis and hemoglobinuria, rats treated with clone 3713 showed significantly reduced levels of free hemoglobin both in the plasma and in the uria.

### REFERENCES

1. Kabat, E. A., et al. Sequences of Proteins of Immunological Interest. 5 ed. NIH National Technical Information Service. (1991) 1-3242.
2. Lefranc, M.-P., et al. IMGT®, the international ImMunoGeneTics information System® 25 years on. Nucl. Acids Res. (2015) 43 (D1): D413-D422
3. Lin, K., et al. Development of an anti-human complement C6 monoclonal antibody that inhibits the assembly of membrane attack complexes. Blood Adv. (2020) 4:2049-2057.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, medicine, and molecular biology or related fields are intended to be within the scope of the following claims.

We claim:

1. A composition comprising a human C6 binding molecule, wherein said human C6 binding molecule comprises:
a) a heavy chain variable region, wherein said heavy chain variable region comprises:
i) a CDRH1 amino acid sequence comprising SEQ ID NO: 14, 22, 30, 38, or 46, or any of the preceding with one with one or two conservative amino acid changes,
ii) a CDRH2 amino acid sequence comprising SEQ ID NO:15, 23, 31, 39, or 47, or any of the preceding with one or two conservative amino acid changes, and
iii) a CDRH3 amino acid sequence comprising SEQ ID NO: 16, 24, 32, 40, or 48, or any of the preceding with one with one or two conservative amino acid changes, and/or;
b) a light chain variable region, wherein said light chain variable region comprises;
i) a CDRL1 amino acid sequence comprising SEQ ID NO: 18, 26, 34, 42, or 50, or any of the preceding with one with one or two conservative amino acid changes,
ii) a CDRL2 amino acid sequence comprising SEQ ID NO: 19, 27, 35, 43, or 51, or any of the preceding with one with one or two conservative amino acid changes, and
iii) a CDRL3 amino acid sequence comprising SEQ ID NO: 20, 28, 36, 44, or 52, or any of the preceding with one with one or two conservative amino acid changes.

2. The composition of claim 1, wherein said human C6 binding molecule is an antibody, minibody, diabody, scFv, or antibody fragment capable of binding human C6.

3. The composition of claim 2, wherein said antibody fragment is a Fab, F(ab′)2 or Fv antibody fragment.

4. The composition of claim 2, wherein said antibody or antibody fragment comprises at least an antigen binding portion of: 1) C601 HFKC4 HuG4K (aka 3713) antibody, 2) C601 HFKI HuG4K antibody, 3) C601 HPKI HuG4K antibody, 4) C601 HOKC4 HuG4K antibody, or 5) C601 HPKC4 HuG4K antibody.

5. The composition of claim 1, wherein said heavy chain and/or light chain variable region comprises a human framework region.

6. The composition of claim 1, wherein said human C6 binding molecule further comprises a light chain constant region and a CH1 heavy chain constant region.

7. The composition of claim 6, wherein said C6 binding molecule further comprises a CH2 heavy chain constant region and/or a CH3 heavy chain constant region.

8. The composition of claim 7, wherein said light chain constant region is human or a humanized murine, and/or wherein said CH1, CH2, and CH3 heavy chain constant regions are human or are humanized murine.

9. The composition of claim 1, wherein said human C6 binding molecule is an antibody or antigen binding portion thereof that has an Fc region characterized in that it: i) has an Fc cellular binding site; or ii) has a Fc complement binding site.

10. The composition of claim 1, wherein said human C6 binding molecule comprises an antibody, wherein the light chain constant region of said antibody is selected from: IgG Kappa and IgG Lambda, and wherein the heavy chain constant region of said antibody is selected from: IgG1, IgG2, IgG3, and IgG4.

11. The composition of claim 1, wherein said human C6 binding molecule comprises an antibody, or antigen binding portion thereof, which is glycosylated or non-glycosylated.

12. The composition of claim 1, further comprising a physiologically tolerable buffer.

13. The composition of claim 1, wherein said heavy chain variable regions comprises SEQ ID NO: 13, 21, 29, 37, or 45, or any of the preceding with one or more conservative amino acid changes.

14. The composition of claim 1, wherein said light chain variable region comprises SEQ ID NO: 17, 25, 33, 41, or 49, or any of the preceding with one or more conservative amino acid changes.

15. A composition comprising at least one of the following:
a) a first nucleic acid sequence encoding a heavy chain variable region, wherein said heavy chain variable region comprises:
i) a CDRH1 amino acid sequence comprising SEQ ID NO: 14, 22, 30, 38, or 46, or any of the preceding with one with one or two conservative amino acid changes,
ii) a CDRH2 amino acid sequence comprising SEQ ID NO:15, 23, 31, 39, or 47, or any of the preceding with one or two conservative amino acid changes, and
iii) a CDRH3 amino acid sequence comprising SEQ ID NO: 16, 24, 32, 40, or 48, or any of the preceding with one with one or two conservative amino acid changes, and/or;
b) a second nucleic acid sequence encoding a light chain variable region, wherein said light chain variable region comprises:
i) a CDRL1 amino acid sequence comprising SEQ ID NO: 18, 26, 34, 42, or 50, or any of the preceding with one with one or two conservative amino acid changes,
ii) a CDRL2 amino acid sequence comprising SEQ ID NO: 19, 27, 35, 43, or 51, or any of the preceding with one with one or two conservative amino acid changes, and
iii) a CDRL3 amino acid sequence comprising SEQ ID NO: 20, 28, 36, 44, or 52, or any of the preceding with one with one or two conservative amino acid changes.

16. The composition of claim 15, further comprising an expression vector, and wherein said first and/or second nucleic acid sequences are present in said expression vector.

17. The composition of claim 15, wherein said light and heavy chain variable regions are part of a human C6 binding molecule.

18. The composition of claim 17, wherein said human C6 binding molecule is an antibody, minibody, diabody, scFv, or antibody fragment capable of binding human C6.

19. The composition of claim 18, wherein said antibody fragment is a F(ab′)2, Fab or Fv antibody fragment.

20. The composition of claim 18, wherein said antibody or antibody fragment comprises at least an antigen binding portion of: 1) C601 HFKC4 HuG4K (aka clone 3713) antibody, 2) C601 HFKI HuG4K antibody, 3) C601 HPKI HuG4K antibody, 4) C601 HOKC4 HuG4K antibody, or 5) C601 HPKC4 HuG4K antibody.

21. The composition of claim 15, wherein said light and/or heavy chain variable region comprises a human framework region.

22. The composition of claim 17, wherein said human C6 binding molecule further comprises a light chain constant region and a CH1 heavy chain constant region.

23. The composition of claim 22, wherein said human C6 binding molecule further comprises a CH2 heavy chain constant region.

24. The composition of claim 23, wherein said human C6 binding molecule further comprises a CH3 heavy chain constant region.

25. The composition of claim 24, wherein said light chain constant region is human or humanized murine, and/or wherein said CH1, CH2, and CH3 heavy chain constant regions are human or are humanized murine.

26. The composition of claim 17, wherein said human C6 binding molecule comprises an antibody, wherein the light chain constant region of said antibody is selected from: IgG Kappa and IgG Lambda, and wherein the heavy chain constant region of said antibody is selected from: IgG1, IgG2, IgG3, and IgG4.

27. The composition of claim 15, wherein said heavy chain variable regions comprises SEQ ID NO: 13, 21, 29, 37, or 45, or any of the preceding with one or more conservative amino acid changes.

28. The composition of claim 15, wherein said light chain variable region comprises SEQ ID NO: 17, 25, 33, 41, or 49, or any of the preceding with one or more conservative amino acid changes.

29. A method of treating or preventing a complement-mediated disease comprising:
treating a subject with a human C6 binding molecule as recited in any of claims 1-14, or an expression vector encoding said human C6 binding molecule or said anti-C6 antibody, and
wherein said subject has, or is suspected to develop, a complement-mediated disease.

30. The method of claim 29, wherein said complement-mediated disease is selected from the group consisting of: membrane-proliferative glomerulonephritis, cold agglutinin disease, catastrophic antiphospholipid syndrome, antibody-mediated transplantation rejection, ischemia/reperfusion injury, rheumatoid arthritis, atherosclerosis, and Guillain Barre Syndrome.

31. The method of claim 29, wherein said complement-mediated disease is selected from the group consisting of: myasthenia gravis, paroxysmal nocturnal hemoglobinuria, atypical hemolytic uremic syndrome, neuromyelitis optica spectrum disorders, and dense-deposit disease.

32. The method of claim 29, wherein said human C6 binding molecule is an antibody or antigen binding portion thereof.

33. The method of claim 32, wherein said antibody or antigen binding portion thereof is a human antibody or antigen binding portion thereof.

34. The method of claim 32, wherein said antibody or antigen binding portion thereof is a humanized antibody or antigen binding portion thereof.

35. The method of claim 29, wherein said heavy chain variable regions comprises SEQ ID NO: 13, 21, 29, 37, or 45, or any of the preceding with one or more conservative amino acid changes.

36. The method of claim 29, wherein said light chain variable region comprises SEQ DD NO: 17, 25, 33, 41, or 49, or any of the preceding with one or more conservative amino acid changes.
